(19) United States
(12) Patent Application Publication
(10) Pub. No.: US 2023/0346976 A1
(43) Pub. Date: Nov. 2, 2023

(54) MUSCLE-SPECIFIC HYBRID PROMOTER

(71) Applicant: Aldevron, L.L.C., South Fargo, ND (US)

(72) Inventor: James A. Williams, Lincoln, NE (US)

(73) Assignee: Aldevron, L.L.C., South Fargo, ND (US)

(21) Appl. No.: 18/181,796

(22) Filed: Mar. 10, 2023

Related U.S. Application Data
(63) Continuation of application No. PCT/US2021/049914, filed on Sep. 10, 2021.
(60) Provisional application No. 63/077,339, filed on Sep. 11, 2020.

(51) Int. Cl. A61K 48/00; C12N 15/85; C12N 15/67

(52) U.S. Cl. CPC A61K 48/0058; C12N 15/85; C12N 15/67; C12Y 207/03002; C12N 2830/15; C12N 2830/008; C12N 2830/50

(57) ABSTRACT

The present disclosure provides novel combinations of muscle-specific enhancers and promoters useful for achieving high and persistent expression in muscle tissue or myocytes. The muscle-specific promoter elements are derived from a desmin promoter. The muscle-specific enhancer elements are derived from a desmin promoter and a muscle creatine kinase enhancer.

### CROSS-REFERENCE TO RELATED APPLICATIONS

The present application is a continuation of International Patent Application Number PCT/US2021/049914, entitled “Muscle-Specific Hybrid Promoter” which was filed filed Sep. 10, 2021, the entire contents of which are hereby incorporated herein by reference in their entirety. International Patent Application Number PCT/US2021/049914 claims priority to U.S. Provisional Application No. 63/077,339, entitled “Muscle-Specific Hybrid Promoter” which was filed Sep. 11, 2020, the entire contents of which are hereby incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 10, 2021, is named 85535-328408_SL.txt and is 48,920 bytes in size.

### BACKGROUND

Ubiquitous promoters, such as CMV, EF1 or CAG, do not allow targeted expression of a gene product. This can result in adverse side effects associated with expression in non-target tissues. For example, expression in antigen presenting cells can lead to an untoward immune response against the transgene (Weeratna R D, Wu T, Efler S M, Zhange L, Davis H L, 2001 Gene Ther. 8:1872).

In muscle tissue, the use of a muscle-specific expression vector can avoid the off-target expression problem, however, the low transgene expression levels from vectors containing muscle-specific promoters limits the cell and gene therapy applications of these vectors.

Thus, there is a need for muscle-specific promoters and enhancer elements that can be incorporated into muscle-specific expression vectors for cell and gene therapy.

### SUMMARY

The present disclosure provides compositions and methods for the expression of transgenes in muscle cells using a muscle-specific regulatory nucleic acid sequence.

A primary object of the invention is to provide expression vectors optimized for high level transgene expression in muscle cells and tissue.

A primary object of the invention is to provide expression vectors optimized for sustained transgene expression in muscle cells and tissue.

A primary object of the invention is to provide expression vectors optimized for low transgene expression in non-muscle tissue.

A primary object of the invention is to provide expression vectors optimized for low CpG to GpG dinucleotide ratio.

Another object of the invention is to provide enhancer/promoter combinations that can direct high level and sustained expression levels in muscle cells and tissue using a variety of non-viral and viral expression vector types.

These objects are achieved by combining desmin muscle-specific promoters and desmin muscle-specific enhancers and MCK muscle-specific enhancers to provide hybrid promoters that drive transgene expression in muscle cells and tissues. The resulting hybrid promoters are useful for muscle cell and gene therapy. The various muscle-specific hybrid promoters of the invention may be used for muscle-specific transgene expression in cultured cells or tissues from, by way of example but not limitation, episomal or integrated plasmid, Nanoplasmid, minicircle, Doggybone, MIDGE, adenoviral, adeno-associated viral (AAV), retroviral, and lentiviral vectors.

In some embodiments, a muscle-specific regulatory nucleic acid sequence is provided that includes a mammalian desmin promoter, a mammalian desmin enhancer, and one or more mammalian muscle creatine kinase (MCK) enhancers that are operably linked.

In some embodiments, a vector comprising the muscle-specific regulatory nucleic acid sequences of the present disclosure is provided.

In some embodiments, a host cell comprising a vector of the present disclosure is provided.

In some embodiments, a method for expressing a transgene in a eukaryotic cell includes the step of transfecting the eukaryotic cell with a vector of the present disclosure.

In some embodiments, a method for replicating a vector of the present disclosure is provided that includes the step of transforming a host cell with a vector of the present disclosure and incubating the cell under conditions sufficient to replicate the vector.

### BRIEF DESCRIPTION OF THE DRAWINGS

For a more complete understanding of the present invention and the advantages thereof, reference is now made to the following description taken in conjunction with the accompanying drawings.

FIG. 1A depicts a vector map of the NTC8685-EGFP Nanoplasmid.

FIG. 1B depicts a vector map of the NTC8685-3×MCKenh-CMV-EGFP Nanoplasmid.

FIG. 1C depicts a vector map of the NTC8685-3×MCKenh-MCAT-CMV-EGFP Nanoplasmid.

FIG. 1D depicts a vector map of the NTC8685-C5-C12-EGFP Nanoplasmid.

FIG. 1E depicts a vector map of the NTC8685-3×MCKenh-C5-C12-EGFP Nanoplasmid.

FIG. 1F depicts a vector map of the NTC8685-3×MCKenh-MCAT-C5-C12-EGFP Nanoplasmid.

FIG. 1G depicts a vector map of the NTC8685-3×MCKenh-MCK-EGFP Nanoplasmid.

FIG. 1H depicts a vector map of the NTC8685-3×MCKenh-MCAT-MCK-EGFP Nanoplasmid.

FIG. 1I depicts a vector map of the NTC8685-Desmin-EGFP Nanoplasmid.

FIG. 1J depicts a vector map of the pVAX1-EGFP Nanoplasmid.

FIG. 2A depicts EGFP expression results in HEK 293 cells.

FIG. 2B depicts EGFP expression results in C5-C12 myotubes.

FIG. 3A depicts a vector map of where the muscle-specific regulatory nucleic acid sequence is of SEQ ID NO: 35 and the Nanoplasmid backbone is of SEQ ID NO: 27.

FIG. 3B depicts a vector map of where the muscle-specific regulatory nucleic acid sequence is of SEQ ID NO: 36 and the Nanoplasmid backbone is of SEQ ID NO: 27.

FIG. 3C depicts a vector map of where the muscle-specific regulatory nucleic acid sequence is of SEQ ID NO: 35 and the Nanoplasmid backbone is of SEQ ID NO: 28.

FIG. 3D depicts a vector map of where the muscle-specific regulatory nucleic acid sequence is of SEQ ID NO: 36 and the Nanoplasmid backbone is of SEQ ID NO: 28

FIG. 4A depicts a vector map of where the muscle-specific regulatory nucleic acid sequence is of SEQ ID NO: 35 and the Nanoplasmid backbone is of SEQ ID NO: 28.

FIG. 4B depicts a vector map of where the muscle-specific regulatory nucleic acid sequence is of SEQ ID NO: 35 and the Nanoplasmid backbone is of SEQ ID NO: 28.

FIG. 4C depicts a vector map of where the muscle-specific regulatory nucleic acid sequence is of SEQ ID NO: 36 and the Nanoplasmid backbone is of SEQ ID NO: 28.

FIG. 4D depicts a dual promoter vector map of where the first muscle-specific regulatory nucleic acid sequence is of SEQ ID NO: 35 for the heavy chain and the second muscle-specific regulatory nucleic acid sequence is of SEQ ID NO: 36 for the light chain and the Nanoplasmid backbone is of SEQ ID NO: 28.

FIG. 5A depicts a vector map of where the muscle-specific regulatory nucleic acid sequence is of SEQ ID NO: 56 and the Nanoplasmid backbone is of SEQ ID NO: 27.

FIG. 5B depicts a vector map of where the muscle-specific regulatory nucleic acid sequence is of SEQ ID NO: 56 and the Nanoplasmid backbone is of SEQ ID NO: 28.

FIG. 5C depicts a vector map of where the muscle-specific regulatory nucleic acid sequence is of SEQ ID NO: 57 and the Nanoplasmid backbone is of SEQ ID NO: 28.

FIG. 5D depicts a dual-promoter vector map of where the muscle-specific regulatory nucleic acid sequence is of SEQ ID NO: 56 for the heavy chain and the muscle-specific regulatory nucleic acid sequence is of SEQ ID NO: 58 for the light chain and the Nanoplasmid backbone is of SEQ ID NO: 27.

FIG. 6 depicts transfection efficiency as determined by luciferase expression. Nanotaxi® Luciferase plasmid DNA administration in skeletal muscles leads to high luciferase expression. Swiss mice were injected i.m at D0 with 10 μg of the different plasmid DNA formulated with Nanotaxi®. At day 7, injected muscles were harvested and analyzed for their luciferase expression. Symbols represent individual injected muscles and horizontal bars the mean and SEM of a group of five mice injected bilaterally.

### DETAILED DESCRIPTION

The present disclosure provides compositions and methods for the expression of transgenes in muscle cells using a muscle-specific regulatory nucleic acid sequence and methods for replicating vectors containing said muscle-specific regulatory nucleic acid sequences.

### Definitions

As used herein, the singular forms “a”, “an” and “the” include plural referents unless the context clearly dictates otherwise.

The use of the term “or” in the claims and the present disclosure is used to mean “and/or” unless explicitly indicated to refer to alternatives only or the alternatives are mutually exclusive.

Use of the term “about”, when used with a numerical value, is intended to include +/−10%. By way of example but not limitation, if a number of nucleotides is identified as about 200, this would include 180 to 220 (plus or minus 10%).

As used herein “cmv” or “CMV” refers to cytomegalovirus.

As used herein “lentiviral vector” refers to an integrative viral vector that can infect dividing and non-dividing cells. Also call Lentiviral transfer plasmid. Plasmid encodes Lentiviral LTR flanked expression unit. Transfer plasmid is transfected into production cells along with Lentiviral envelope and packaging plasmids required to make viral particles.

As used herein “lentiviral envelope vector” refers to a plasmid encoding envelope glycoprotein.

As used herein “lentiviral packaging vector” refers to one or two plasmids that express gag, pol and Rev gene functions required to package the lentiviral transfer vector.

As used herein “minicircle” refers to covalently closed circular plasmid derivatives in which the bacterial region has been removed from the parent plasmid by in vivo or in vitro site-specific recombination or in vitro restriction digestion/ligation. Minicircle vectors are replication incompetent in bacterial cells.

As used herein “Nanoplasmid™ vector” or “Nanoplasmid” refers to a vector combining an RNA selectable marker with a bacterial replication origin, such as a R6K, ColE2 or ColE2-related replication origin. For example, Nanoplasmid vectors can include, by way of example, but not limitation, NTC9385C, NTC9685C, NTC9385R, NTC9685R vectors and modifications described in WO 2014/035457.

As used herein “NTC8 series” refers to vectors, such as NTC8385, NTC8485 and NTC8685 plasmids are antibiotic-free pUC origin vectors that contain a short RNA (RNA-OUT) selectable marker instead of an antibiotic resistance marker such as kanR. The creation and application of these RNA-OUT based antibiotic-free vectors are described in WO2008/153733.

As used herein “retroviral vector” refers to integrative viral vector that can infect dividing cells. Also call transfer plasmid. Plasmid encodes Retroviral LTR flanked expression unit. Transfer plasmid is transfected into production cells along with envelope and packaging plasmids required to make viral particles.

As used herein “retroviral envelope vector” refers to a plasmid encoding envelope glycoprotein.

As used herein “retroviral packaging vector” refers to a plasmid that encodes retroviral gag and pol genes required to package the retroviral transfer vector.

As used herein “transfection” or “transformation” refers to a method to deliver nucleic acids into cells [e.g. poly(lactide-co-glycolide) (PLGA), ISCOMs, liposomes, niosomes, virosomes, block copolymers, Pluronic block copolymers, chitosan, and other biodegradable polymers, microparticles, microspheres, calcium phosphate nanoparticles, nanoparticles, nanocapsules, nanospheres, poloxamine nanospheres, electroporation, nucleofection, piezoelectric permeabilization, sonoporation, iontophoresis, ultrasound, SQZ high speed cell deformation mediated membrane disruption, corona plasma, plasma facilitated delivery, tissue tolerable plasma, laser microporation, shock wave energy, magnetic fields, contactless magneto-permeabilization, gene gun, microneedles, microdermabrasion, hydrodynamic delivery, high pressure tail vein injection, etc] as known in the art and included herein by reference.

As used herein “transgene” refers to a gene of interest that is cloned into a vector for expression in a target organism.

As used herein “vector” refers to a gene delivery vehicle, including viral (e.g. Alphavirus, Poxvirus, Lentivirus, Retrovirus, Adenovirus, Adenovirus related virus, etc.) and non-viral (e.g. plasmid, MIDGE, transcriptionally active PCR fragment, minicircles, bacteriophage, Nanoplasmid™, etc.) vectors. These are well known in the art and are included herein by reference.

A “Doggybone” as referred to herein is a minimal, closed-linear DNA construct that is an enzymatically produced capped linear vector.

A “MIDGE” as referred to herein is a minimalistic, immunologically defined gene expression vector that is a small, linear, covalently closed, dumbbell-shaped molecule.

To determine percent sequence identity, as understood in the present disclosure, a query sequence (e.g. a nucleic acid sequence) is aligned to one or more subject sequences using any suitable sequence alignment program that is well known in the art, for instance, the computer program ClustalW (version 1.83, default parameters), which allows alignments of nucleic acid sequences to be carried out across their entire length (global alignment). Chema et al., 2003 Nucleic Acids Res., 31:3497-500. In a preferred method, the sequence alignment program (e.g. ClustalW) calculates the best match between a query and one or more subject sequences, and aligns them so that identities, similarities, and differences can be determined. Gaps of one or more nucleotides can be inserted into a query sequence, a subject sequence, or both, to maximize sequence alignments.

Muscle-Specific Regulatory Nucleic Acid Sequences

In some embodiments, a muscle-specific regulatory nucleic acid sequence is provided that includes a mammalian desmin promoter, a mammalian desmin enhancer, and one or more mammalian muscle creatine kinase (MCK) enhancers that are operably linked.

In any of the foregoing embodiments, the mammalian desmin promoter, mammalian desmin enhancer and one or more MCK enhancers can be human or murine. It should be understood that the mammalian desmin promoter, mammalian desmin enhancer and one or more MCK enhancers can be derived from any mammalian species as these nucleic acid sequences can be determined using known methods and search tools. By way of example, but not limitation, the mammalian desmin promoter, mammalian desmin enhancer and one or more MCK enhancers can be derived from human, murine, equine, porcine, feline, canine, or primate sources. It should be further understood that the origin of each of the mammalian desmin promoter, mammalian desmin enhancer and one or more mammalian MCK enhancers can be different or the same. It should be further understood that where desmin enhancer(s) or multiple mammalian MCK enhancers are included in the muscle-specific regulatory nucleic acid sequence, each of the multiple elements can be from the same or different origin. By way of example, but not limitation, the muscle-specific regulatory nucleic acid sequence can include a mammalian desmin promoter, mammalian desmin enhancer and one or more mammalian MCK enhancers that are all human or murine or any combination of human and murine elements, such as a human desmin promoter, human desmin enhancer and one or more murine MCK enhancers. In preferred embodiments, the muscle-specific regulatory nucleic acid sequence can include one or more murine MCK enhancers in combination with a murine desmin enhancer and a murine desmin promoter or one or more murine MCK enhancers in combination with a human desmin enhancer and a human desmin promoter, more preferably three copies of a murine MCK enhancer in combination with a human desmin enhancer and a human desmin promoter. It should be further understood that the mammalian desmin promoter, mammalian desmin enhancer and one or more mammalian MCK enhancers can be full-length or truncated, so long as the truncation preserves at least a portion of the function of the element, e.g. a truncated mammalian desmin promoter would still have promoter activity as assayed by expression of a downstream transgene.

In any of the foregoing embodiments, where the mammalian desmin promoter is human, the mammalian desmin promoter can include a nucleic acid sequence having 80% or more identity to any of SEQ ID NO: 7, SEQ ID NO: 8, SEQ ID NO: 9, and SEQ ID NO: 10. By way of example, but not limitation, the mammalian desmin promoter can have at least 80%, 85%, 90%, 95%, 96%, 97%, 98%, 99% or more identity to any of SEQ ID NO: 7, SEQ ID NO: 8, SEQ ID NO: 9, and SEQ ID NO: 10. In some embodiments, the mammalian desmin promoter comprises the sequence of any of SEQ ID NO: 7, SEQ ID NO: 8, SEQ ID NO: 9, and SEQ ID NO: 10.

In any of the foregoing embodiments, where the mammalian desmin promoter is murine, the mammalian desmin promoter can include a nucleic acid sequence having 80% or more identity to any of SEQ ID NO: 4 and SEQ ID NO: 5. By way of example, but not limitation, the mammalian desmin promoter can have at least 80%, 85%, 90%, 95%, 96%, 97%, 98%, 99% or more identity to any of SEQ ID NO: 4 and SEQ ID NO: 5. In some embodiments, the mammalian desmin promoter comprises the sequence of SEQ ID NO: 4 or SEQ ID NO: 5.

In any of the foregoing embodiments, the mammalian desmin promoter can include an INR sequence. By way of example, but not limitation, the INR sequence, which includes an initiator element, can be the nucleic acid sequence of SEQ ID NO: 59. By way of further example, but not limitation, the INR sequence can include the nucleic acid sequence tataaaa and the nucleic acid sequence yyanwyy separated by an intervening sequence and, optionally, comprising a downstream sequence downstream of yyanwyy. It should be understood that the initatior element can include the consensus sequence of yyanwyy, which can, by way of example, be tcagtcc. By way of still further example, but not limitation, the intervening sequence can be from about 20 to about 25 nucleotides in length, such as about 20, 21, 22, 23, 24, or 25 nucleotides. By way of still further example, but not limitation, the downstream sequence can be of any suitable length.

In any of the foregoing embodiments, the muscle-specific regulatory nucleic acid sequence can include more than one mammalian desmin enhancer. By way of example, the muscle-specific regulatory nucleic acid sequence can include 1, 2, 3, 4, 5 or more mammalian desmin enhancer sequences. In some embodiments, the muscle-specific regulatory nucleic acid sequence includes only one mammalian desmin enhancer, i.e. the muscle-specific regulatory nucleic acid sequence does not include more than one mammalian desmin enhancer.

In any of the foregoing embodiments, the mammalian desmin enhancer can include a nucleic acid sequence having at least 80% identity to SEQ ID NO: 3 or SEQ ID NO: 6. By way of example, but not limitation, the mammalian desmin enhancer can have at least 80%, 85%, 90%, 95%, 96%, 97%, 98%, 99% or more identity to SEQ ID NO: 3 or SEQ ID NO: 6. In some embodiments, the mammalian desmin enhancer comprises the sequence of SEQ ID NO: 3. In some embodiments, the mammalian desmin enhancer comprises the sequence of SEQ ID NO: 6.

In any of the foregoing embodiments, the muscle-specific regulatory nucleic acid sequence can include one or more mammalian MCK enhancers. By way of example, but not limitation, the muscle-specific regulatory nucleic acid sequence can include two or more mammalian MCK enhancers, 1 to 3 mammalian MCK enhancers, 1, 2, 3, 4, 5 or more mammalian MCK enhancers. It should be understood that the one or more mammalian MCK enhancers can be separated by linking sequences or can have other elements between them if there are more than one, e.g. by the desmin enhancer or desmin promoter, or by the transgene. By way of example, but not limitation, the one or more mammalian MCK enhancers can be separated by 1000, 900, 800, 700, 600, 500, 450, 400, 350, 300, 250, 200, 150, 100, 90, 80, 70, 60, 50, 40, 30, 20, 10, 9, 8, 7, 6, 5, 4, 3, 2, 1 or 0 nucleotides. It should be further understood that the one or more mammalian MCK enhancers can be separated by the mammalian desmin promoter or mammalian desmin enhancer(s).

In any of the foregoing embodiments, each of the one or more mammalian MCK enhancers can include a nucleic acid sequence having at least 80% identity to SEQ ID NO: 11 (a murine MCK enhancer) or SEQ ID NO: 12 (a human MCK enhancer). By way of example, but not limitation, the one or more mammalian MCK enhancers can have at least 80%, 85%, 90%, 95%, 96%, 97%, 98%, 99% or more identity to SEQ ID NO: 11 or SEQ ID NO: 12. In some embodiments, the one or more mammalian MCK enhancers each comprise the sequence of SEQ ID NO: 11 or SEQ ID NO: 12. In some embodiments, each of the one or more mammalian MCK enhancers can be a MCK CK7 enhancer such as, by way of example, but not limitation, the MCK CK7 enhancer of SEQ ID NO: 1. By way of further example, but not limitation, the one or more mammalian MCK enhancers can have at least 80%, 85%, 90%, 95%, 96%, 97%, 98%, 99% or more identity to SEQ ID NO: 1.

In any of the foregoing embodiments, the muscle-specific regulatory nucleic acid sequence can further include one or more additional enhancers. In some embodiments, the one or more additional enhancers each comprise a nucleic sequence having 80% or more identity to any of SEQ ID NO: 13, SEQ ID NO: 14, SEQ ID NO: 15, and SEQ ID NO: 16. By way of example, but not limitation, each of the one or more additional enhancers can have at least 80%, 85%, 90%, 95%, 96%, 97%, 98%, 99% or more identity to any of SEQ ID NO: 13, SEQ ID NO: 14, SEQ ID NO: 15, and SEQ ID NO: 16. In some embodiments, the one or more additional enhancers each comprise the sequence of any of SEQ ID NO: 13, SEQ ID NO: 14, SEQ ID NO: 15, and SEQ ID NO: 16.

In some embodiments, the muscle-specific regulatory nucleic acid sequence includes, as its only enhancers, the mammalian desmin enhancer and the one or more mammalian MCK enhancers. In some embodiments, the muscle-specific regulatory nucleic acid sequence does not include one or more additional enhancers. In some embodiments, the muscle-specific regulatory nucleic acid sequence does not include a vertebrate troponin I IRE (FIRE) enhancer.

In any of the foregoing embodiments, the muscle-specific regulatory nucleic acid sequence can further include an intron. In any of the foregoing embodiments, the intron can be positioned 3′ to the mammalian desmin promoter. Any suitable intron can be used. In any of the foregoing embodiments, the intron can include a nucleic acid sequence having 80% or more identity to SEQ ID NO: 17 or SEQ ID NO: 18. By way of example, but not limitation, the intron can have at least 80%, 85%, 90%, 95%, 96%, 97%, 98%, 99% or more identity to SEQ ID NO: 17 or SEQ ID NO: 18. In some embodiments, the intron comprises the sequence of SEQ ID NO: 17 or SEQ ID NO: 18. In any of the foregoing embodiments, the intron can have a size of about 100 to about 10,000 nucleotides. By way of example, but not limitation, the intron can have a size of about 100 to about 10,000 nucleotides, about 100 to about 9,000 nucleotides, about 100 to about 8,000 nucleotides, about 100 to about 7,000 nucleotides, about 100 to about 6,000 nucleotides, about 100 to about 5,000 nucleotides, about 100 to about 4,000 nucleotides, about 100 to about 3,000 nucleotides, about 100 to about 2,000 nucleotides, about 100 to about 1,000 nucleotides, about 100 to about 500 nucleotides, about 100 to about 400 nucleotides, about 100 to about 300 nucleotides, about 100 to about 200 nucleotides, about 200 to about 10,000 nucleotides, about 200 to about 9,000 nucleotides, about 200 to about 8,000 nucleotides, about 200 to about 7,000 nucleotides, about 200 to about 6,000 nucleotides, about 200 to about 5,000 nucleotides, about 200 to about 4,000 nucleotides, about 200 to about 3,000 nucleotides, about 200 to about 2,000 nucleotides, about 200 to about 1,000 nucleotides, about 200 to about 500 nucleotides, about 200 to about 400 nucleotides, about 200 to about 300 nucleotides, about 300 to about 10,000 nucleotides, about 300 to about 9,000 nucleotides, about 300 to about 8,000 nucleotides, about 300 to about 7,000 nucleotides, about 300 to about 6,000 nucleotides, about 300 to about 5,000 nucleotides, about 300 to about 4,000 nucleotides, about 300 to about 3,000 nucleotides, about 300 to about 2,000 nucleotides, about 300 to about 1,000 nucleotides, about 300 to about 500 nucleotides, about 300 to about 400 nucleotides, about 400 to about 10,000 nucleotides, about 400 to about 9,000 nucleotides, about 400 to about 8,000 nucleotides, about 400 to about 7,000 nucleotides, about 400 to about 6,000 nucleotides, about 400 to about 5,000 nucleotides, about 400 to about 4,000 nucleotides, about 400 to about 3,000 nucleotides, about 400 to about 2,000 nucleotides, about 400 to about 1,000 nucleotides, about 400 to about 500 nucleotides, about 500 to about 10,000 nucleotides, about 500 to about 9,000 nucleotides, about 500 to about 8,000 nucleotides, about 500 to about 7,000 nucleotides, about 500 to about 6,000 nucleotides, about 500 to about 5,000 nucleotides, about 500 to about 4,000 nucleotides, about 500 to about 3,000 nucleotides, about 500 to about 2,000 nucleotides, about 500 to about 1,000 nucleotides, about 1,000 to about 10,000 nucleotides, about 1,000 to about 9,000 nucleotides, about 1,000 to about 8,000 nucleotides, about 1,000 to about 7,000 nucleotides, about 1,000 to about 6,000 nucleotides, about 1,000 to about 5,000 nucleotides, about 1,000 to about 4,000 nucleotides, about 1,000 to about 3,000 nucleotides, about 1,000 to about 2,000 nucleotides, about 2,000 to about 10,000 nucleotides, about 2,000 to about 9,000 nucleotides, about 2,000 to about 8,000 nucleotides, about 2,000 to about 7,000 nucleotides, about 2,000 to about 6,000 nucleotides, about 2,000 to about 5,000 nucleotides, about 2,000 to about 4,000 nucleotides, about 2,000 to about 3,000 nucleotides, about 3,000 to about 10,000 nucleotides, about 3,000 to about 9,000 nucleotides, about 3,000 to about 8,000 nucleotides, about 3,000 to about 7,000 nucleotides, about 3,000 to about 6,000 nucleotides, about 3,000 to about 5,000 nucleotides, about 3,000 to about 4,000 nucleotides, about 4,000 to about 10,000 nucleotides, about 4,000 to about 9,000 nucleotides, about 4,000 to about 8,000 nucleotides, about 4,000 to about 7,000 nucleotides, about 4,000 to about 6,000 nucleotides, about 4,000 to about 5,000 nucleotides, about 5,000 to about 10,000 nucleotides, about 5,000 to about 9,000 nucleotides, about 5,000 to about 8,000 nucleotides, about 5,000 to about 7,000 nucleotides, about 5,000 to about 6,000 nucleotides, about 6,000 to about 10,000 nucleotides, about 6,000 to about 9,000 nucleotides, about 6,000 to about 8,000 nucleotides, about 6,000 to about 7,000 nucleotides, about 7,000 to about 10,000 nucleotides, about 7,000 to about 9,000 nucleotides, about 7,000 to about 8,000 nucleotides, about 8,000 to about 10,000 nucleotides, about 8,000 to about 9,000 nucleotides, about 9,000 to about 10,000 nucleotides, or about 100, 200, 300, 400, 500, 1,000, 2,000, 3,000, 4,000, 5,000, 6,000, 7,000, 8,000, 9,000, or 10,000 nucleotides.

In any of the foregoing embodiments, the muscle-specific regulatory nucleic acid sequence can further include a transgene. In any of the foregoing embodiments, the transgene can be positioned 3′ to the mammalian desmin promoter. The transgene can be under the control of the mammalian desmin promoter. The transgene can be any suitable transgene, for example, the transgene can be a therapeutic transgene such as VEGF, a gene therapy replacement gene such as factor IX, a reverse vaccination antigen such as insulin for diabetes, or a therapeutic antibody such as Avastin. In any of the foregoing embodiments, the mammalian desmin promoter can be separated from the transgene by about 500 nucleotides or less, non-inclusive of any intron between the mammalian desmin promoter and the transgene. By way of example, but not limitation, the mammalian desmin promoter can be separated from the transgene by about 0 to about 1000 nucleotides, about 1 to about 1000 nucleotides, about 1 to about 900 nucleotides, about 1 to about 800 nucleotides, about 1 to about 700 nucleotides, about 1 to about 600 nucleotides, about 1 to about 500 nucleotides, about 1 to about 400 nucleotides, about 1 to about 300 nucleotides, about 1 to about 200 nucleotides, about 1 to about 100 nucleotides, about 1 to about 90 nucleotides, about 1 to about 80 nucleotides, about 1 to about 70 nucleotides, about 1 to about 60 nucleotides, about 1 to about 50 nucleotides, about 1 to about 40 nucleotides, about 1 to about 30 nucleotides, about 1 to about 20 nucleotides, about 1 to about 10 nucleotides, about 10 to about 1000 nucleotides, about 10 to about 900 nucleotides, about 10 to about 800 nucleotides, about 10 to about 700 nucleotides, about 10 to about 600 nucleotides, about 10 to about 500 nucleotides, about 10 to about 400 nucleotides, about 10 to about 300 nucleotides, about 10 to about 200 nucleotides, about 10 to about 100 nucleotides, about 100 to about 1000 nucleotides, about 100 to about 500 nucleotides, about 200 to about 1000 nucleotides, about 200 to about 500 nucleotides, about 500 to about 1000 nucleotides, or about 1000, 900, 800, 700, 600, 500, 400, 300, 250, 200, 150, 100, 90, 80, 70, 60, 50, 40, 30, 20, 10, 9, 8, 7, 6, 5, 4, 3, 2, 1 or 0 nucleotides.

In any of the foregoing embodiments, the one or more mammalian MCK enhancers can be positioned 5′ to the mammalian desmin enhancer which can be positioned 5′ to the mammalian desmin promoter. It should be understood that in any of the foregoing embodiments, as already described with respect to the one or more MCK enhancers, there can be linking sequences between the elements of the muscle-specific regulatory nucleic acid sequence so long as the elements are operably linked. By way of example, but not limitation, the one or elements—mammalian desmin promoter, mammalian desmin enhancer and one or more MCK enhancers—of the muscle-specific regulatory nucleic acid sequence can be separated by about 0 to about 1000 nucleotides, about 1 to about 1000 nucleotides, about 1 to about 900 nucleotides, about 1 to about 800 nucleotides, about 1 to about 700 nucleotides, about 1 to about 600 nucleotides, about 1 to about 500 nucleotides, about 1 to about 400 nucleotides, about 1 to about 300 nucleotides, about 1 to about 200 nucleotides, about 1 to about 100 nucleotides, about 1 to about 90 nucleotides, about 1 to about 80 nucleotides, about 1 to about 70 nucleotides, about 1 to about 60 nucleotides, about 1 to about 50 nucleotides, about 1 to about 40 nucleotides, about 1 to about 30 nucleotides, about 1 to about 20 nucleotides, about 1 to about 10 nucleotides, about 10 to about 1000 nucleotides, about 10 to about 900 nucleotides, about 10 to about 800 nucleotides, about 10 to about 700 nucleotides, about 10 to about 600 nucleotides, about 10 to about 500 nucleotides, about 10 to about 400 nucleotides, about 10 to about 300 nucleotides, about 10 to about 200 nucleotides, about 10 to about 100 nucleotides, about 100 to about 1000 nucleotides, about 100 to about 500 nucleotides, about 200 to about 1000 nucleotides, about 200 to about 500 nucleotides, about 500 to about 1000 nucleotides, or about 1000, 900, 800, 700, 600, 500, 400, 300, 250, 200, 100, 90, 80, 70, 60, 50, 40, 30, 20, 10, 9, 8, 7, 6, 5, 4, 3, 2, 1 or 0 nucleotides.

Preferred sequences for the muscle-specific regulatory nucleic acid sequence can include SEQ ID NO: 31, SEQ ID NO: 32, SEQ ID NO: 33, SEQ ID NO: 34, SEQ ID NO: 35, SEQ ID NO: 36, SEQ ID NO: 55, SEQ ID NO: 56, SEQ ID NO: 57 and SEQ ID NO: 58. By way of example, but not limitation, the muscle-specific regulatory nucleic acid sequence can include a nucleic acid sequence having at least 80%, 85%, 90%, 95%, 96%, 97%, 98%, 99% or more identity to any of SEQ ID NO: 31, SEQ ID NO: 32, SEQ ID NO: 33, SEQ ID NO: 34, SEQ ID NO: 35, SEQ ID NO: 36, SEQ ID NO: 55, SEQ ID NO: 56, SEQ ID NO: 57 and SEQ ID NO: 58.

Details of these preferred sequences are provided below:

MCK CK7 enhancer, hDesmin enhancer, hDesmin promoter, MVM intron (SEQ ID NO: 31).

MCK CK7 enhancer, hDesmin enhancer, hDesmin INR promoter, MVM intron (SEQ ID NO: 32).

MCK CK7 enhancer, hDesmin enhancer, hDesminS promoter, MVM intron (SEQ ID NO: 33).

MCK CK7 enhancer, hDesmin enhancer, hDesminS INR promoter, MVM intron (SEQ ID NO: 34).

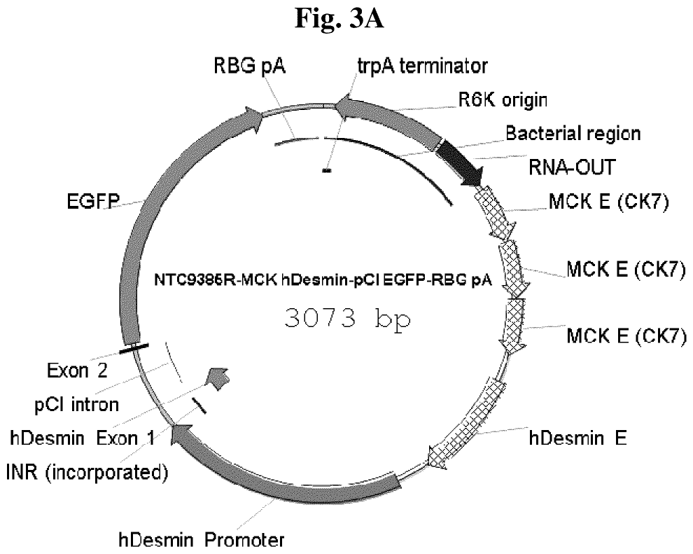

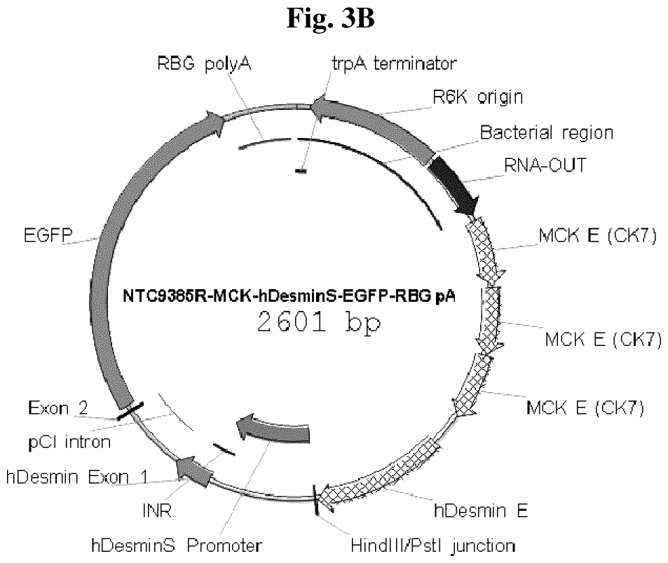

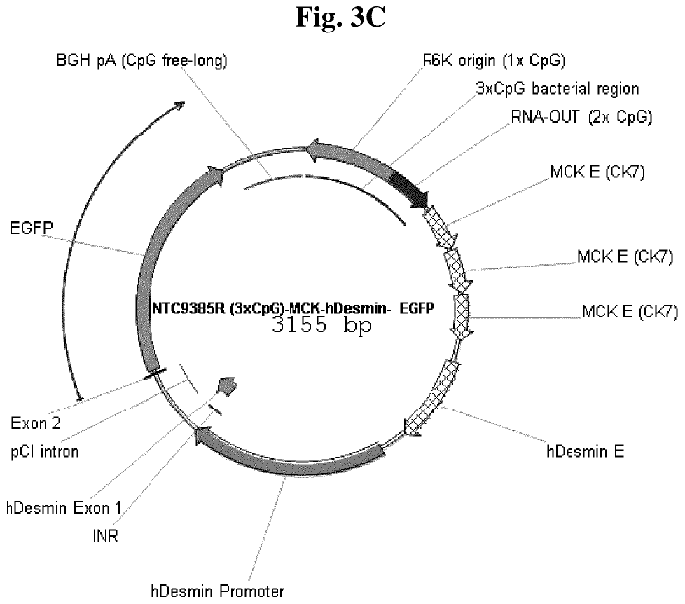

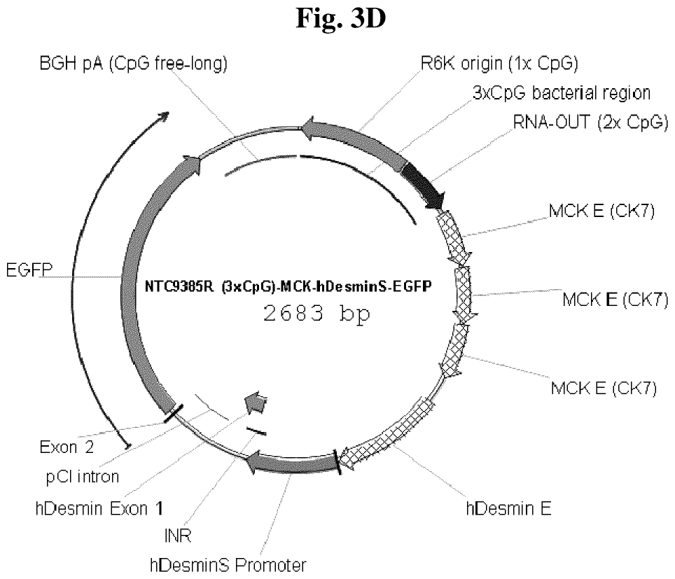

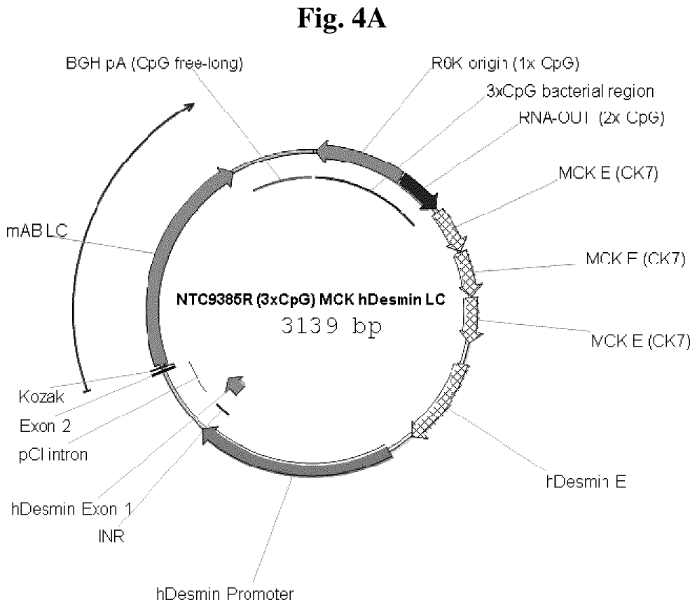

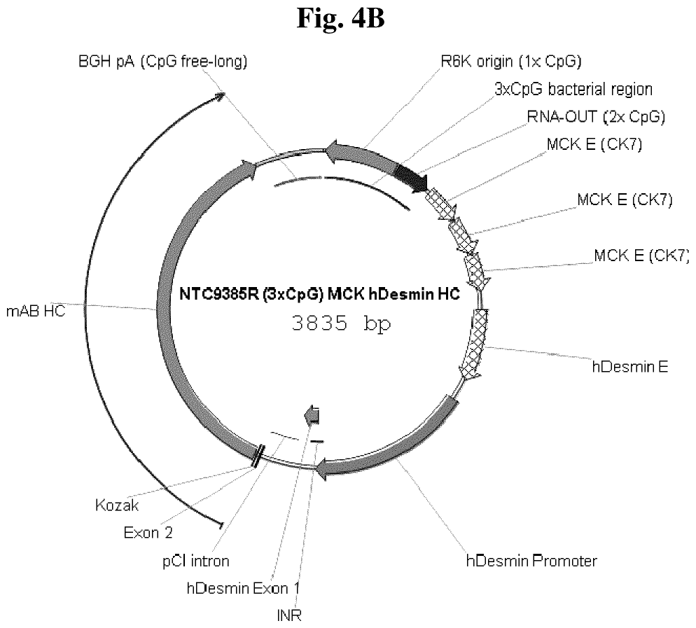

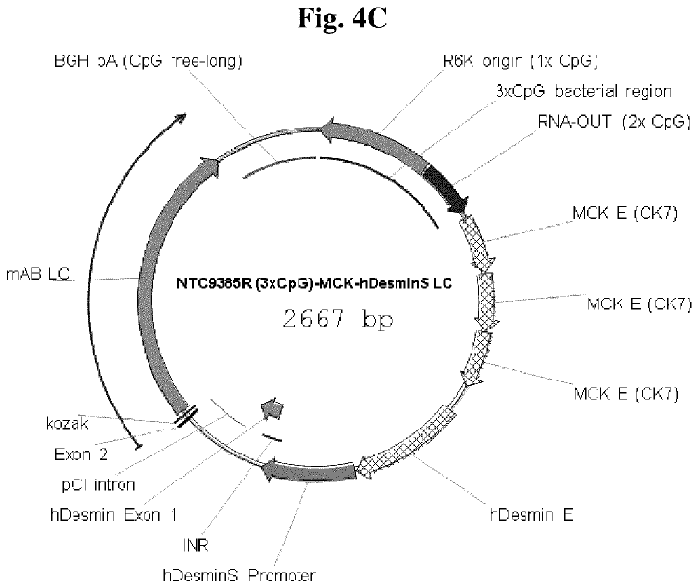

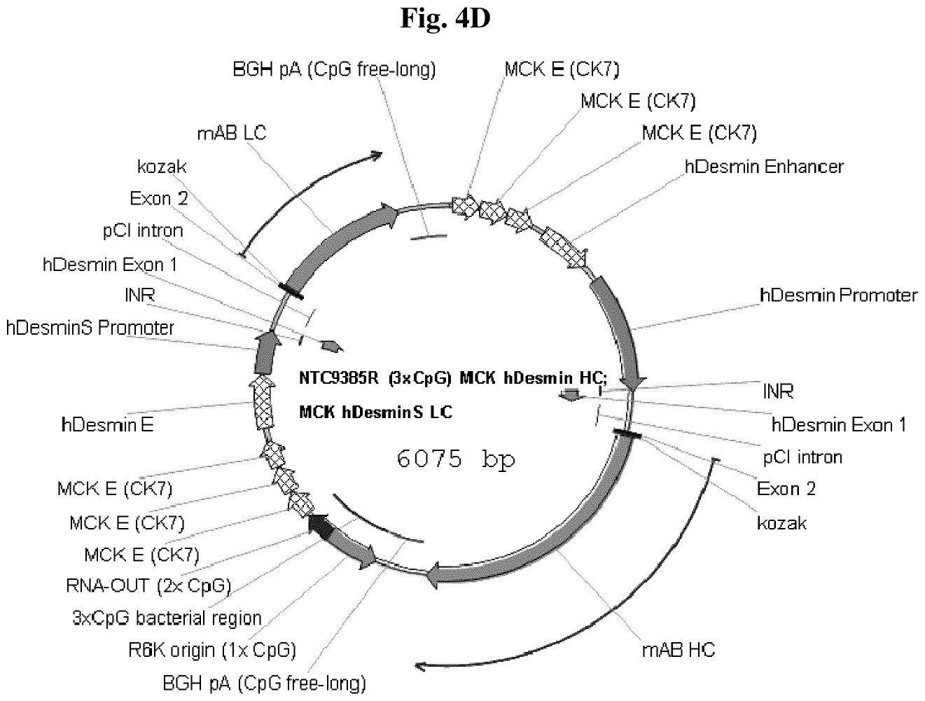

MCK CK7 enhancer, hDesmin enhancer, hDesmin INR promoter, pCI intron (FIGS. 3A, 3C, 4A, 4B and 4D, SEQ ID NO: 35)

MCK CK7 enhancer, hDesmin enhancer, hDesminS INR promoter, pCI intron (FIGS. 3B, 3D, 4C and 4D, SEQ ID NO: 36).

MCK CK7 enhancer, mDesmin enhancer, mDesmin promoter, MVM intron (SEQ ID NO: 55).

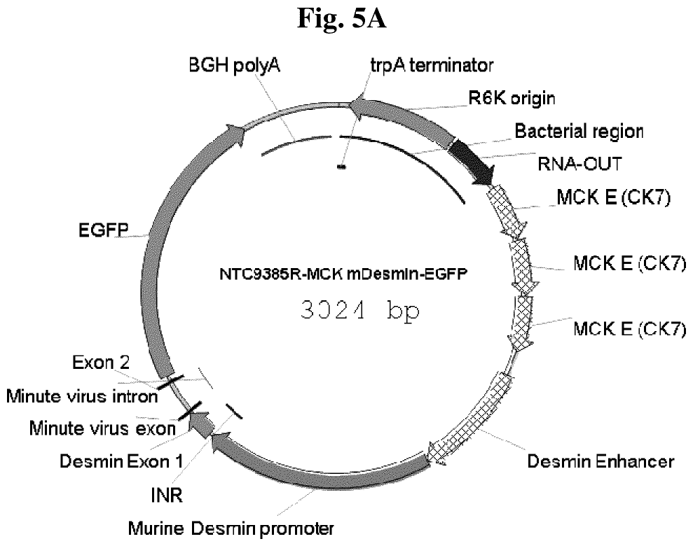

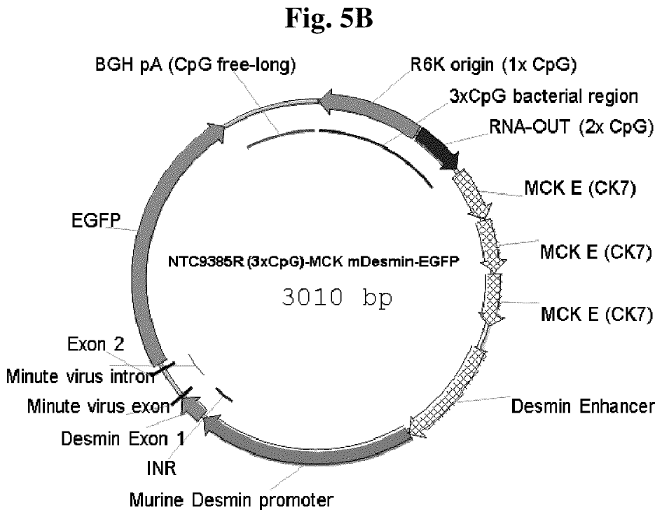

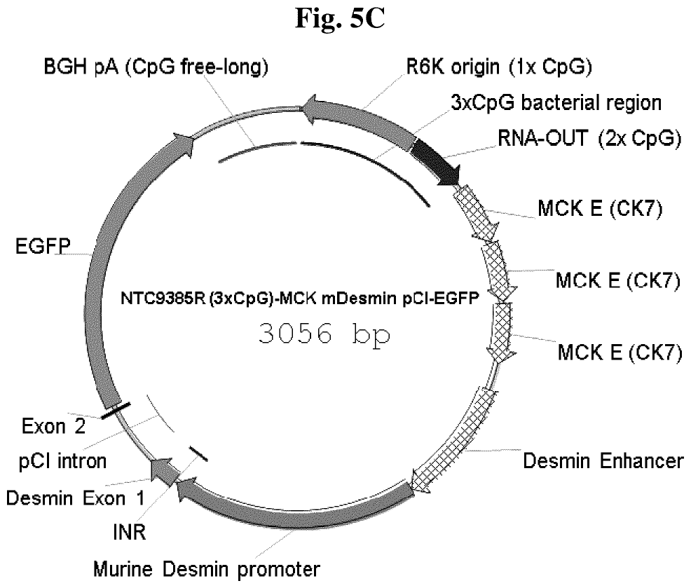

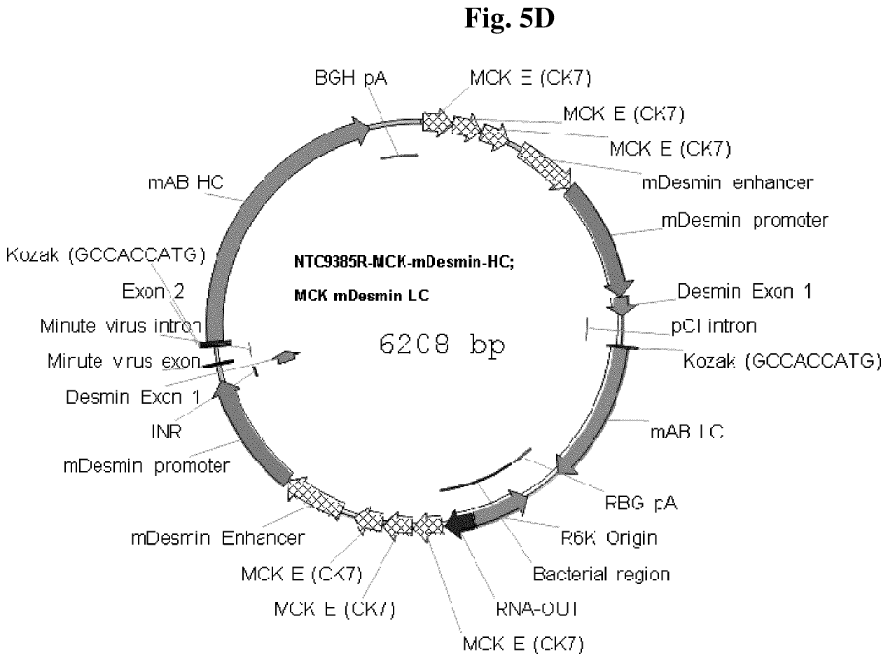

MCK CK7 enhancer, mDesmin enhancer, mDesmin INR promoter, MVM intron (FIGS. 5A, 5B and 5D, SEQ ID NO: 56).

MCK CK7 enhancer, mDesmin enhancer, mDesmin INR promoter, pCI intron (FIG. 5C, (SEQ ID NO: 57).

MCK CK7 enhancer, mDesmin enhancer, mDesmin promoter, pCI intron (FIG. 5D, SEQ ID NO: 58).

A preferred configuration can include, by way of example but not limitation:

5′-one or more Mammalian MCK enhancer(s), mammalian desmin enhancer(s), mammalian desmin promoter-3′.

Other configurations of the muscle-specific regulatory nucleic acid sequence can include, by way of example, but not limitation:

One or more mammalian MCK enhancer(s), mammalian desmin enhancer, one or more mammalian MCK enhancer(s), mammalian desmin promoter.

Mammalian desmin enhancer, one or more mammalian MCK enhancer(s), mammalian desmin promoter.

Mammalian desmin promoter, one or more mammalian MCK enhancer(s), mammalian desmin enhancer.

Mammalian desmin promoter, one or more mammalian MCK enhancer(s), mammalian desmin enhancer, one or more mammalian MCK enhancer(s).

Mammalian desmin promoter, mammalian desmin enhancer, one or more mammalian MCK enhancer(s).

In any of the foregoing other configurations, an intron can be inserted in the muscle-specific regulatory nucleic acid sequence. Similarly, in any of the foregoing configurations, a transgene can be positioned downstream from the mammalian desmin promoter, possibly between the additional elements or between the desmin promoter and the additional elements. It should also be understood that there can be multiple mammalian desmin enhancers and that any combination of the mammalian desmin enhancer(s) and the one or more mammalian MCK enhancers can be made in terms of the order of the elements.

Vectors

In some embodiments, a vector comprising the muscle-specific regulatory nucleic acid sequences of any of the foregoing embodiments is provided.

The vector can be any suitable vector for transfecting a cell with the muscle-specific regulatory nucleic acid sequence. By way of example, but not limitation, the vector can be a plasmid, a minicircle, a Doggybone, a MIDGE, a Nanoplasmid, or a viral vector. By way of further example, but not limitation, the vector can be an episomal non replicative expression vector, an episomal replicative expression vector, a transposon integration vector, a viral integration vector, or a homology directed repair vector.

In some embodiments, where the vector is a viral vector, the viral vector can be an adenovirus, an adeno-associated virus (AAV), a lentivirus or a retrovirus.

In some embodiments, the vector can include more than one muscle-specific regulatory nucleic acid sequences.

In some embodiments, the vector can be a dual promoter vector.

It should be understood that where the vector is a Nanoplasmid, the Nanoplasmid includes a eukaryotic region, which can include the muscle-specific regulatory nucleic acid sequence(s) and transgenes, having 5′ and 3′ ends and a spacer region of less than 500 base pairs that links the 5′ and 3′ ends of the eukaryotic region and which includes a bacterial replication origin such as, by way of example, but not limitation, R6K or ColE2, and a RNA selectable marker. Non-limiting, exemplary R6K origins are provided in SEQ ID NOs: 19-23 and exemplary RNA selectable markers are provided in SEQ ID NOs: 24 and 26. It should be understood that, where the bacterial replication origin is an R6K origin of any one of SEQ ID NOs: 19-23, the bacterial replication origin can have at least 80%, 85%, 90%, 91%, 92%, 93%, 94%, 95%, 96%, 97%, 98%, 99% or 100% identity to the any one of SEQ ID NOs: 19-23, respectively. It should be further understood that, where the RNA selectable marker is one of SEQ ID NO: 24 or SEQ ID NO: 26, the RNA selectable marker can have at least 80%, 85%, 90%, 91%, 92%, 93%, 94%, 95%, 96%, 97%, 98%, 99% or 100% identity to the any one of SEQ ID NOs: 24 or 26, respectively. Nanoplasmid vectors are also described in International Patent Application Publication No. WO 2014/077866 and U.S. Patent Application No. 2010/0184158, each of which is incorporated by reference herein in its entirety.

In some embodiments, where the vector is a Nanoplasmid, the vector can include the sequence of any of SEQ ID NO: 27, SEQ ID NO: 28, SEQ ID NO: 29, and SEQ ID NO: 30. SEQ ID NOs: 27 and 29 are six and seven R6K origin iteron versions of the NTC9385R backbone, while SEQ ID NOs: 28 and 30 are six and seven R6K origin iteron versions of the NTC9385R (3×CpG) backbone.

Nanoplasmid vector maps using these preferred sequences with an example transgene (EGFP) are shown in FIGS. 3A-3D and 5A-5D. Nanoplasmid vector maps using these preferred sequences with an example monoclonal antibody light chain (mAB LC) or heavy chain (mAB HC) or both LC and HC transgenes are shown in FIGS. 4A-D and 5A-D. These vectors can be used for passive immunotherapy, for example for in vivo expression of a virus neutralizing antibody (Bakker J M, Bleeker W K, Parren P W H I. 2004. Mol Ther 10:1525; Tjelle T E, Corthay A, Lunde E, Sandlie I, Michaelsen T E, Mathiesen I, Bogen B. 2004. Mol Ther 9:328; Hollevoet K, Declerck P J. 2017. J Transl Med 15:131). Antibody light and heavy chains may be expressed in different vectors, or both may be expressed in a dual promoter vector. An example dual promoter vector, expressing both LC and HC transgenes from preferred muscle promoters in a single vector are shown in FIGS. 4D and 5D.

In some preferred embodiments, the vector can have a CpG to GpG ratio of less than 0.7. By way of example, but not limitation, the vector can have a CpG to GpG ratio of less than 0.7, 0.65, 0.6, 0.55, 0.5, 0.45, 0.4, 0.35, or 0.3. By way of further example, but not limitation, the vector can have a CpG to GpG ratio of about 0.3 to about 0.7, about 0.3 to about 0.6, about 0.4 to about 0.5, about 0.25, about 0.3, about 0.35, about 0.4, about 0.5, about 0.55, or about 0.6.

In any of the foregoing embodiments, the vector can include two or more muscle-specific regulatory nucleic acid sequences according to any of the foregoing embodiments. By way of example, but not limitation, the vector can include a first muscle-specific regulatory nucleic acid sequence where a first transgene is under control of the mammalian desmin promoter of the first muscle-specific regulatory nucleic acid sequence and a second muscle-specific regulatory nucleic acid sequence where a second transgene is under control of the mammalian desmin promoter of the second muscle-specific regulatory nucleic acid sequence. In such an embodiments, the first and second transgene can encode the same product or different products. By way of further example, but not limitation, the first transgene can encode an antibody heavy chain and the second transgene can encode an antibody light chain.

It should be understood that in any of the foregoing embodiments, the muscle-specific regulatory nucleic acid sequence can be positioned 5′ or 3′ of the transgene(s). By way of example, but not limitation, in a Nanoplasmid, the muscle-specific regulatory nucleic acid sequence could be at a 5′ end of the eukaryotic region or a 3′ end of a eukaryotic region, such as 5′ of or 3′ of the transgene, respectively, as the transgene can still be under the control of the mammalian desmin promoter across the spacer region.

It should likewise be understood that the mammalian MCK enhancers and desmin enhancers of the present disclosure can be positioned downstream of the transgene.

Host Cells

In some embodiments, a transformed host cell comprising a vector of the present disclosure is provided. The host cell can be any suitable bacterial cell, such as DH5α. In some embodiments, a transfected eukaryotic cell comprising a vector of the present disclosure is provided. By way of example, but not limitation, the eukaryotic cell can be a human muscle cell, myotube, or myoblast. It should be understood that human muscle cells can be, by way of example, but not limitation skeletal muscle cells, cardiac muscle cells, and diaphragm muscle cells.

Methods for Producing the Vectors of the Present Disclosure

In some embodiments, a method for preparing a muscle-specific expression vector can include providing a vector comprising a non-muscle-specific promoter or a non-desmin promoter, and modifying the vector such that the non-muscle-specific promoter or non-desmin promoter is replaced by a muscle-specific regulatory nucleic acid sequence of the present disclosure. The vector and the muscle-specific regulatory nucleic acid sequence can be as described in any of the foregoing embodiments of the present disclosure.

Methods for Replication or Expression

In some embodiments, a method for expressing a transgene in a eukaryotic cell includes the step of transfecting the eukaryotic cell with a vector of the present disclosure. It should be understood that the transfection can be performed under conditions sufficient for the vector to express the transgene in the eukaryotic cell. In some embodiments, the eukaryotic cell is a muscle cell. It should be understood that human muscle cells can be, by way of example but not limitation, skeletal muscle cells, cardiac muscle cells, or diaphragm muscle cells. By way of example, but not limitation, methods of transforming a host cell with a vector of the present disclosure can include administering to a subject a vector of the present disclosure. By way of further example, but not limitation, the subject can be a human.

In some embodiments, a method for replicating a vector of the present disclosure is provided that includes the step of transforming a host cell with a vector of the present disclosure and incubating the cell under conditions sufficient to replicate the vector. Methods for transfecting a host cell and conditions for incubating the host cell under conditions sufficient to replicate the vector are known to those skilled in the art.

### EXAMPLES

In the following examples, cloning to create vectors containing the various transgenes, muscle promoters, 5′ UTR introns, etc. described here were constructed using standard restriction fragment ligation mediated cloning. All constructs were verified correct by restriction digestion and sequencing.

In the following examples, Nanoplasmid vectors were cloned and propagated in R6K origin ‘copy cutter’ host cell lines NTC1050811-HF and NTC1050811-HF dcm- that were created and disclosed in Williams 2019 VIRAL AND NON-VIRAL NANOPLASMID VECTORS WITH IMPROVED PRODUCTION International Patent Application Publication No. WO2019/183248 which is incorporated herein by reference. pVAX1 vectors were propagated in DH5α cells.

In the following examples, for shake flask production proprietary Plasmid+ shake culture medium was used. The seed cultures were started from glycerol stocks or colonies and streaked onto LB medium agar plates containing 50 μg/mL antibiotic (kanR selection pVAX1 plasmids) or 6% sucrose (for RNA-OUT selection NTC8 plasmids and NTC9 Nanoplasmids). The plates were grown at 30-32° C.; cells were resuspended in media and used to provide approximately 2.5 OD₆₀₀ inoculums for the 500 mL Plasmid+ shake flasks that contained 50 g/mL antibiotic for kanR selection pVAX1 plasmids or 0.5% sucrose to select for RNA-OUT plasmids and Nanoplasmids. Flask were grown with shaking to saturation at the growth temperatures as indicated. Low endotoxin Nanoplasmid DNA was purified using Nucleobond AX 2,000 or AX 10,000 columns (Macherey Nagel, Duren, Germany).

Table 1 summaries various muscle specific promoters described in the art. Native muscle promoters such as human or murine desmin, or murine muscle creatine kinase (MCK) have relatively low expression levels compared to CMV. Many hybrid muscle promoters that combine enhancers and promoters from different muscle specific control elements are also relatively low expression compared to CMV. For example, Souza and Armentano WO2002095006 obtained relatively weak promoters by combining an MCK enhancer with the hDesmin promoter (Table 1; DC310 and DC311). This teaches away from obtaining strong muscle promoters by combining MCK enhancers and hDesmin promoter. Other promoter-enhancer combinations such as tMCK and Sk-CRM4-Des were shown to create hybrid muscle promoters with activities exceeding the CMV promoter (Table 1).

TABLE 1
Expression Characteristics and Structure of Muscle-Specific Constructs

| | | | | | | Skeletal | |
|---|---|---|---|---|---|---|---|
| | | First | Second | | | Muscle | |
| | | Enhancer | Enhancer | | | Expression | |
| Name | Vector | Source | source | Promoter | Intron | level | Reference |
| MCKCK6 | AAV | NA | mMCK2R5S | mMCK −357-+7 | None | 12% CMV | Hauser et |
| | | | (−1262-−1060) | (SEQ ID | | | al 2000. |
| | | | (SEQ ID | NO: 40) | | | Mol Ther |
| | | | NO: 37) | | | | 2: 16 |
| Enh358MCK | AAV | NA | mMCK | mMCK −357-+7 | None | 1/3 CMV | Wang et al |
| | | | (−1262-−1060) | (SEQ ID | | | 2008. |
| | | | (SEQ ID | NO: 40) | | | Gene Ther |
| | | | NO: 11) | | | | 15: 1489 |
| tMCK | AAV | NA | mMCK | mMCK −80-+7 | None | 4x CMV | Wang et al |
| | | | 2R5S (3x) | (SEQ ID | | | 2008. |
| | | | (SEQ ID | NO: 39) | | | Gene Ther |
| | | | NO: 37 | | | | 15: 1489 |
| | | | (3x)) | | | | |
| CK7 | AAV | NA | mMCK | mMCK | None | Comparable | Salva et al |
| | | | CK7 | INR −357-+50 | | to CMV | 2007. Mol |
| | | | (−1262-−1060) | | | | Ther |
| | | | (SEQ ID | | | | 15: 320 |
| | | | NO: 1) | | | | |
| MHCK7 | AAV | α-MHC | mMCK | mMCK | None | Comparable | Salva et al |
| | | | CK7 | INR −357-+50 | | to CMV | 2007. Mol |
| | | | (−1262-−1060) | | | | Ther |
| | | | (SEQ ID | | | | 15: 320 |
| | | | NO: 1) | | | | |
| DC308 | plasmid | NA | hDesmin2x | hDesmin | Intron | <40% | Souza and |
| | | | (−973-−731) | (−228-−+1) | | CMV | Armentano |
| | | | 2x | | | | WO2002095006 |
| DC309 | plasmid | NA | hDesmin4x | hDesmin | Intron | <35% | Souza and |
| | | | (−973-−731) | (−228-−+1) | | CMV | Armentano |
| | | | 4x | | | | Supra |
| DC310 | plasmid | NA | mMCK 2x | hDesmin | Intron | <15% | Souza and |
| | | | (−1256-1051) | (−228-−+1) | | CMV | Armentano |
| | | | 2x | | | | Supra |
| DC311 | plasmid | NA | mMCK 4x | hDesmin | Intron | <15% | Souza and |
| | | | (−1256-1051) | (−228-−+1) | | CMV | Armentano |
| | | | 4x | | | | Supra |
| DC318 | plasmid | hDesmin2x | mMCK 2x | hDesmin | Intron | <50% | Souza and |
| | | (−973-−731) | (−1256-1051) | (−228-−+1) | | CMV | Armentano |
| | | | | | | | Supra |
| Muscle | AAV | mDesmin | mMCK | mMCK | Intron | Comparable | Piekarowicz |
| Hybrid | | (−976-−826) | (−1262-1060) | INR −358-+50+ | with | to CMV | et al |
| (MH) | | | | | MCK | | 2019. |
| | | | | | SIE | | Meth Clin |
| | | | | | | | Dev 15: |
| | | | | | | | 157 |
| Des | AAV | NA | mDesmin | mDesmin | MVM | Comparable | Sarcar et |
| | | | (−895-−592) | (−591-+83) | intron | to CMV | al 2019. |
| | | | (SEQ ID | (SEQ ID | (SEQ | | Nat |
| | | | NO: 3) | NO: 4) | ID NO: | | Comm |
| | | | | | 17) | | 10: 492 |
| Sk- | AAV | SK-CRM4 | mDesmin | mDesmin | MVM | 25-173x | Sarcar et |
| CRM4- | | (myosin | (−895-−592) | (−591-+83) | intron | CMV | al 2019. |
| Des | | light chain | (SEQ ID | (SEQ ID | (SEQ | | Nat |
| | | enhancer) | NO: 3) | NO: 4) | ID NO: | | Comm |
| | | (SEQ ID | | | 17) | | 10: 492 |
| | | NO: 14) | | | | | |
| Sk- | AAV | SK-CRM4 | hDesmin) | hDesmin | MVM | 2-3x | Chuah and |
| CRM4- | | (myosin | (−990-−620) | (−619-+86) | intron | mDesmin | Vanderdriessche |
| hDes1.0 | | light chain | (SEQ ID | (SEQ ID | (SEQ | Sk-CRM4- | WO2018178067 |
| | | enhancer) | NO: 6) | NO: 7) | ID NO: | Des | |
| | | (SEQ ID | | | 17) | | |
| | | NO: 14) | | | | | |
| Sk- | AAV | SK-CRM4 | hDesmin | hDesmin | MVM | 2-3x | Chuah and |
| CRM4- | | (myosin | (−1340-−620) | (−619-+86) | intron | mDesmin | Vanderdriessche |
| hDes1.4 | | light chain | (SEQ ID | (SEQ ID | (SEQ | Sk-CRM4- | WO2018178067 |
| | | enhancer) | NO: 52) | NO: 7) | ID NO: | Des | |
| | | (SEQ ID | | | 17) | | |
| | | NO: 14) | | | | | |
| CRE02 | AAV | CRE02 | hDesmin | hDesmin | MVM | 9-11x | Chuah and |
| Sk- | | (ACTA1) | (−1340-−620) | (−619-+86) | intron | hDesmin | Vanderdriessche |
| CRM4- | | (SEQ ID | (SEQ ID | (SEQ ID | (SEQ | Sk-CRM4- | WO2018178067 |
| hDes1.4 | | NO: 15) + | NO: 52) | NO: 7) | ID NO: | hDes1.4 | |
| | | SK-CRM4 | | | 17) | | |
| | | (myosin | | | | | |
| | | light chain | | | | | |
| | | enhancer) | | | | | |
| | | (SEQ ID | | | | | |
| | | NO: 14) | | | | | |
| CRE64 | AAV | CRE64 | hDesmin | hDesmin | MVM | 9-15x | Chuah and |
| Sk- | | (ATP2A1) | (−1340-−620) | (−619-+86) | intron | hDesmin | Vanderdriessche |
| CRM4- | | (SEQ ID | (SEQ ID | (SEQ ID | (SEQ | Sk-CRM4- | WO2018178067 |
| hDes1.4 | | NO: 16) + | NO: 52) | NO: 7) | ID NO: | hDes1.4 | |
| | | SK-CRM4 | | | 17) | | |
| | | (myosin | | | | | |
| | | light chain | | | | | |
| | | enhancer) | | | | | |
| | | (SEQ ID | | | | | |
| | | NO: 14) | | | | | |

### Example 1: EGFP Reporter Expression in Various Promoter-Enhancer Constructs

Various native and hybrid muscle promoter version of the NTC8685 vector (containing the pUC origin and antibiotic free RNA-OUT sucrose selection cassette) (Luke, J M, Vincent J M, Du, S X, Gerdemann U, Leen A M, Whalen R G, Hodgson C P Williams J A. 2011. Gene Ther 18:334) were constructed and expression levels determined in C2C12 myotubes, A549 and HEK293 cells.

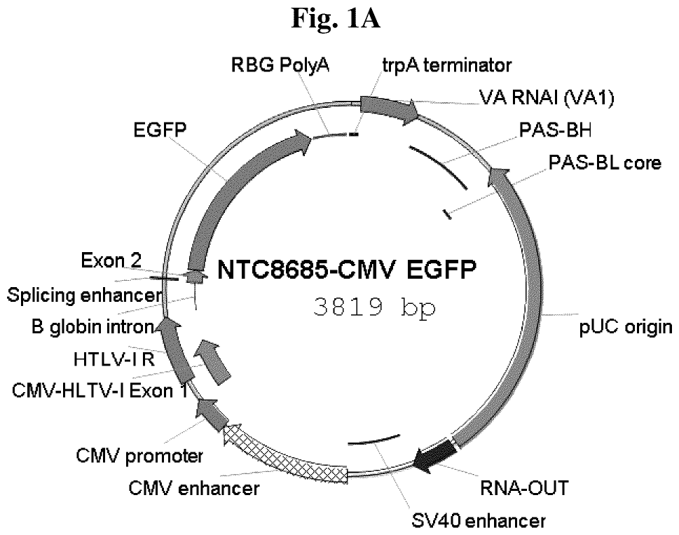

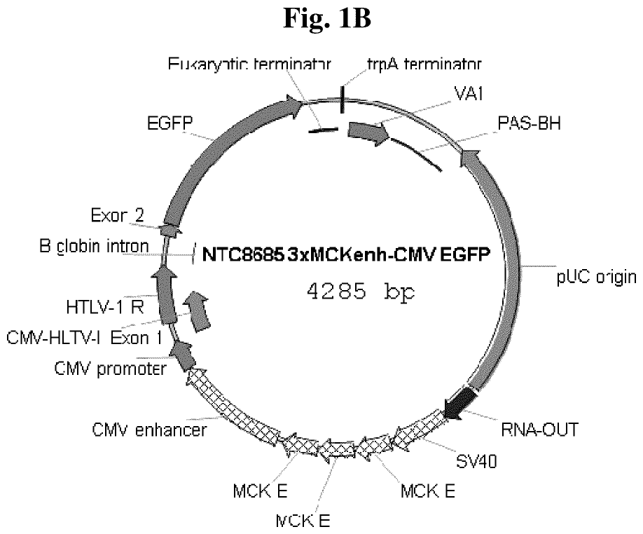

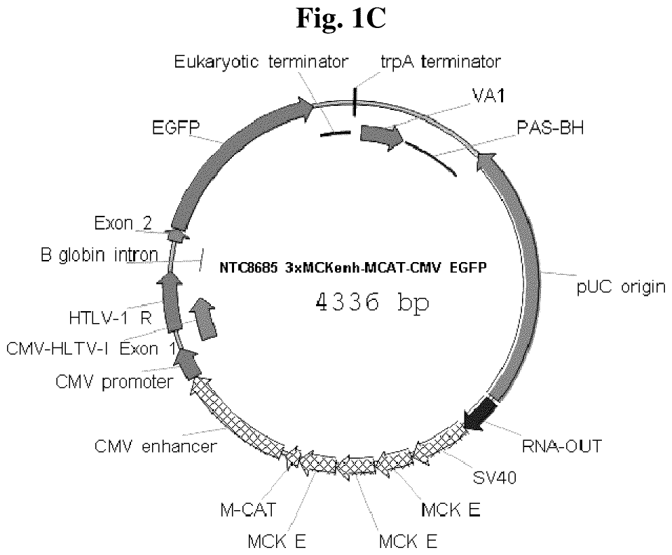

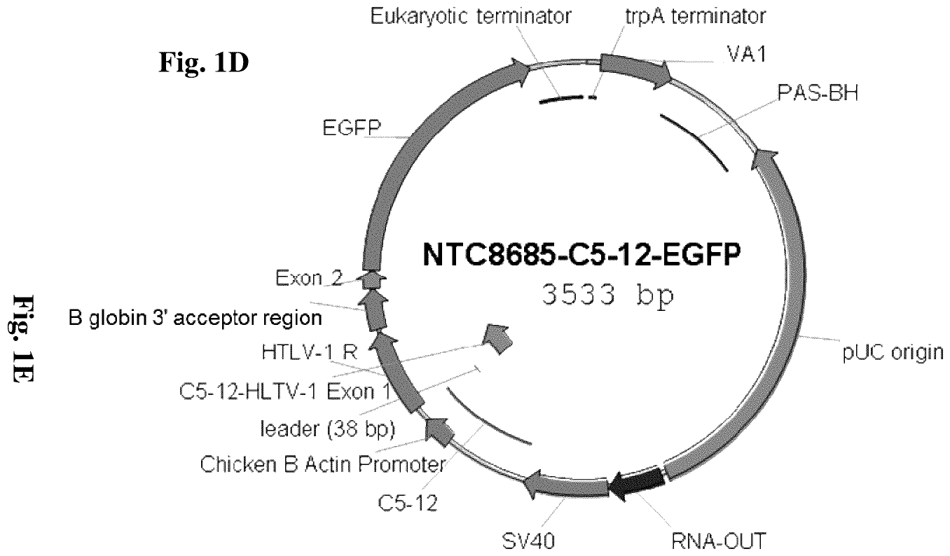

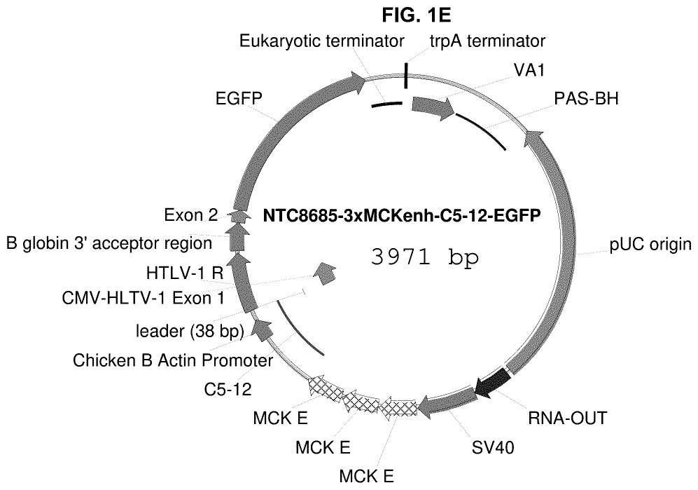

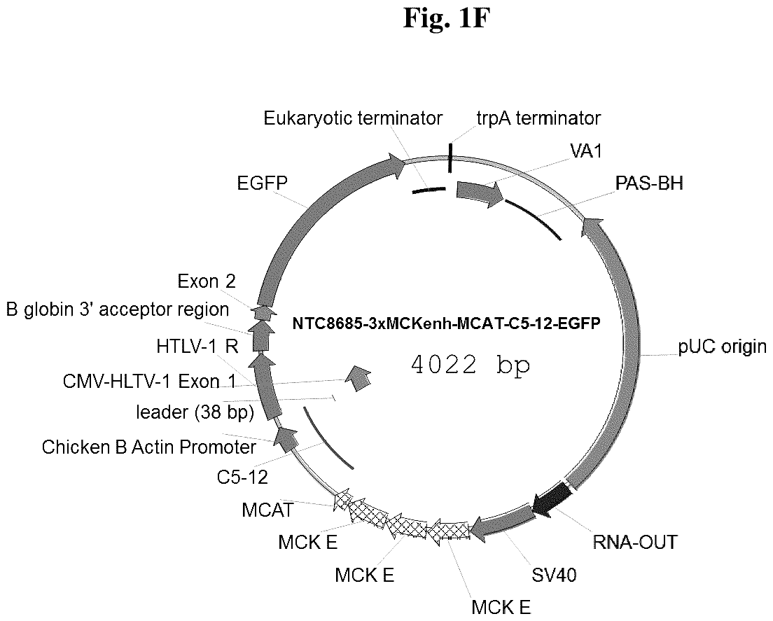

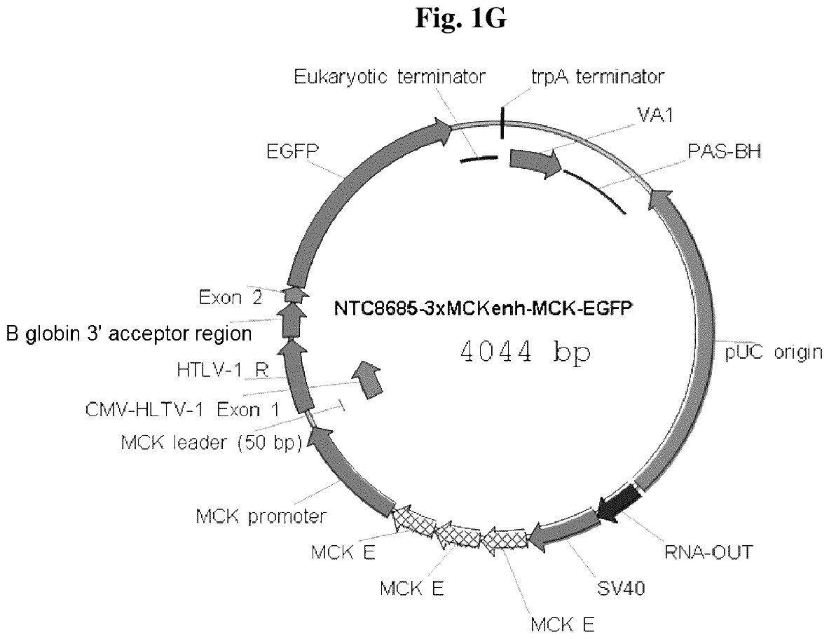

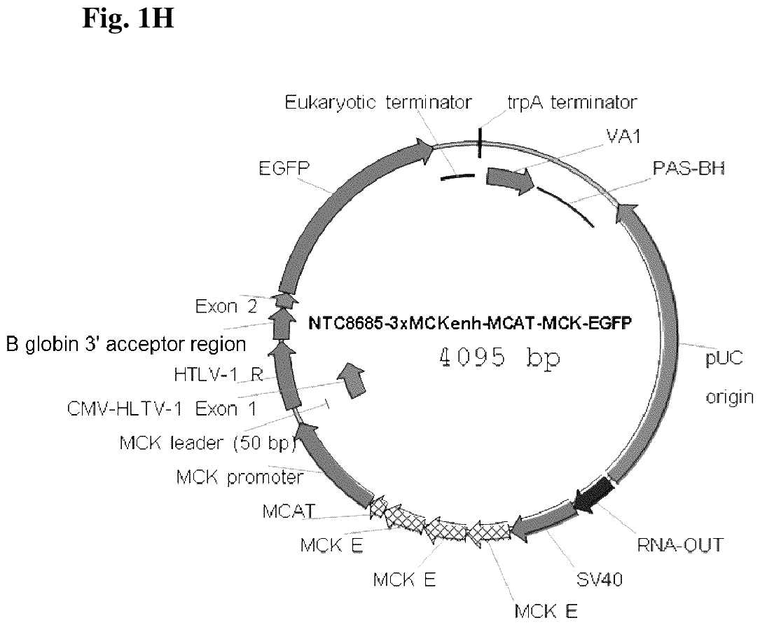

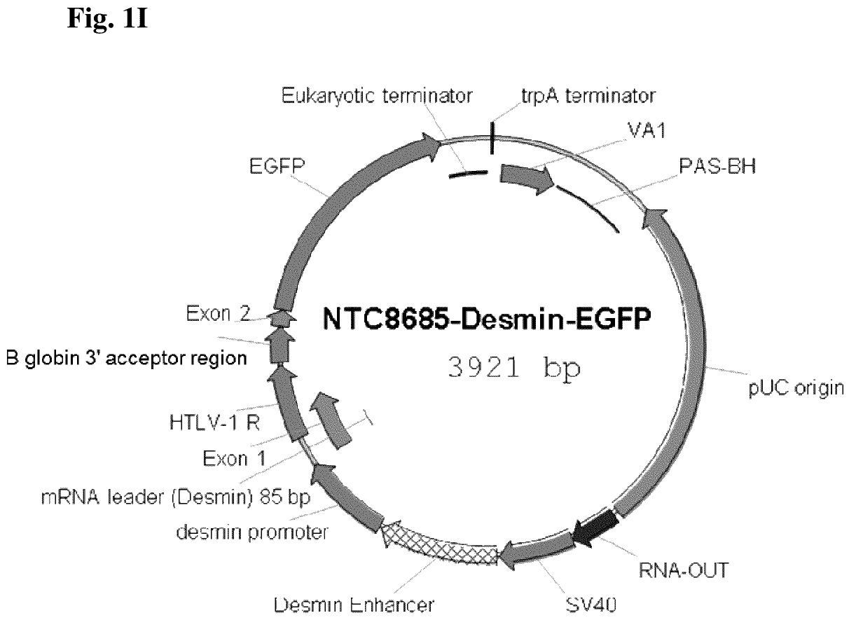

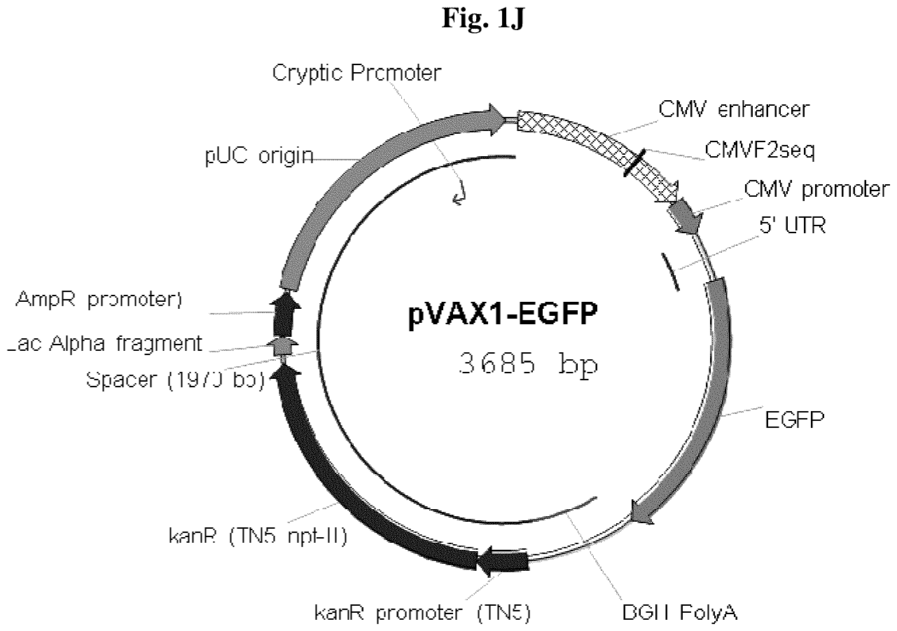

Vector maps for the constructs tested are provided in FIGS. 1A-H. The individual sequence of certain elements of the vectors are listed and described in Table 2 below:

TABLE 2
Sequence Information for Vector Maps

| Element | Sequence |
|---|---|
| SV40 Enhancer | SEQ ID NO: 44 |
| HR β Intron | SEQ ID NO: 53 |
| CMV Enhancer-Promoter-Exon 1 | SEQ ID NO: 45 |
| (FIGS. 1A, B, and C) | |
| Murine Muscle Creatine Kinase CK7 | SEQ ID NO: 1 |
| (MCK E) | |
| 3x MCK E | SEQ ID NO: 2 |
| MCAT (2x) | SEQ ID NO: 47 |
| C5-C12 Enhancer-Promoter-Exon 1 | SEQ ID NO: 48 |
| Murine MCK Promoter Exon 1 | SEQ ID NO: 40 |
| (mMCK promoter (−357-+7) | |
| Human Desmin Enhancer (hDesmin | SEQ ID NO: 50 |
| (−1008-−558) Enhancer) | |
| hDesmin Promoter-Exon 1 (−253-+86) | SEQ ID NO: 49 |
| CMV Enhancer-Promoter-Exon 1 | SEQ ID NO: 51 |
| (FIG. 1 J) | |

Adherent HEK293 (human embryonic kidney), A549 (human lung carcinoma) and C2C12 (Mus musculus, mouse muscle), cell lines were obtained from the American Type Culture Collection (Manassas, VA, USA). Cell lines were propagated in Dulbecco's modified Eagle's medium/F12 containing 10% fetal bovine serum and split (0.25% trypsin-EDTA) using Invitrogen (Carlsbad, CA, USA) reagents and conventional methodologies.

For transfections, cells were plated on 24-well tissue culture dishes. Plasmids and Nanoplasmids were transfected into cell lines using Lipofectamine 2000 following the manufacturer's instructions (Invitrogen). For HEK293 and A549 cells, 0.1 ug test plasmid per well was used in the transfection and expression was determined 2-3 days after transfection. For C2C12, 0.4 ug test plasmid per well was used in the transfection after which cells were differentiated into myotubes by addition of DMEM F12+2% Horse serum (differentiation media). Expression in differentiated myotube structures was determined on T=6-7 days.

Total cellular lysates for EGFP determination were prepared by resuspending cells in cell lysis buffer (CelLytic M, Sigma, St Louis, MO, USA), lysing cells by incubating for 30 min at 37° C., followed by a freeze-thaw cycle at −80° C. Lysed cells were clarified by centrifugation and the supernatants assayed for EGFP by FLX800 microplate fluorescence reader (Bio-Tek, Winooski, VT, USA).

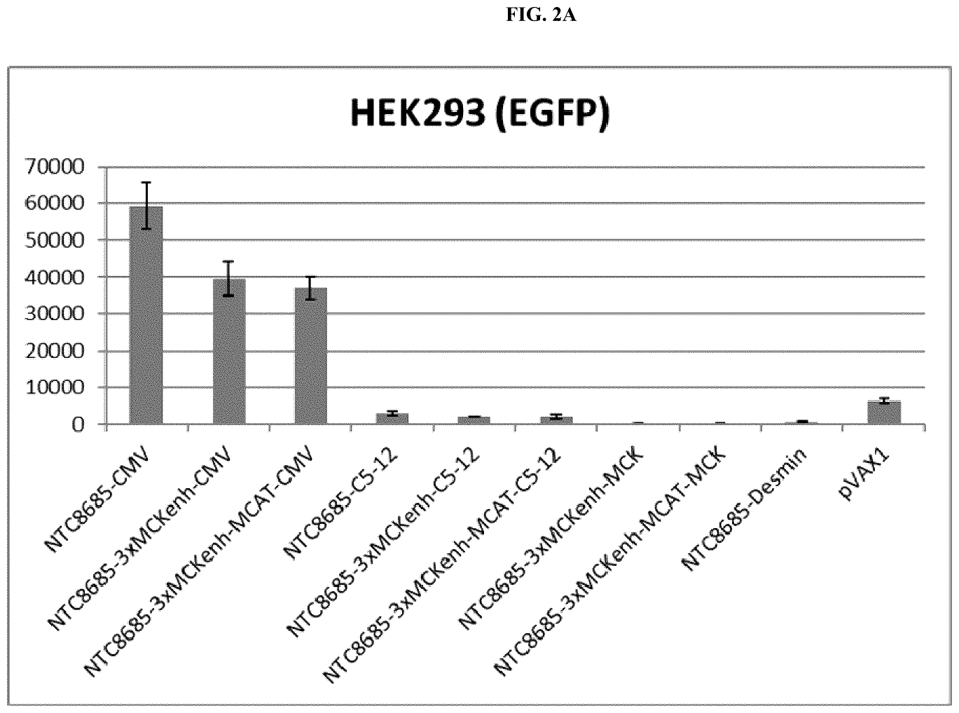

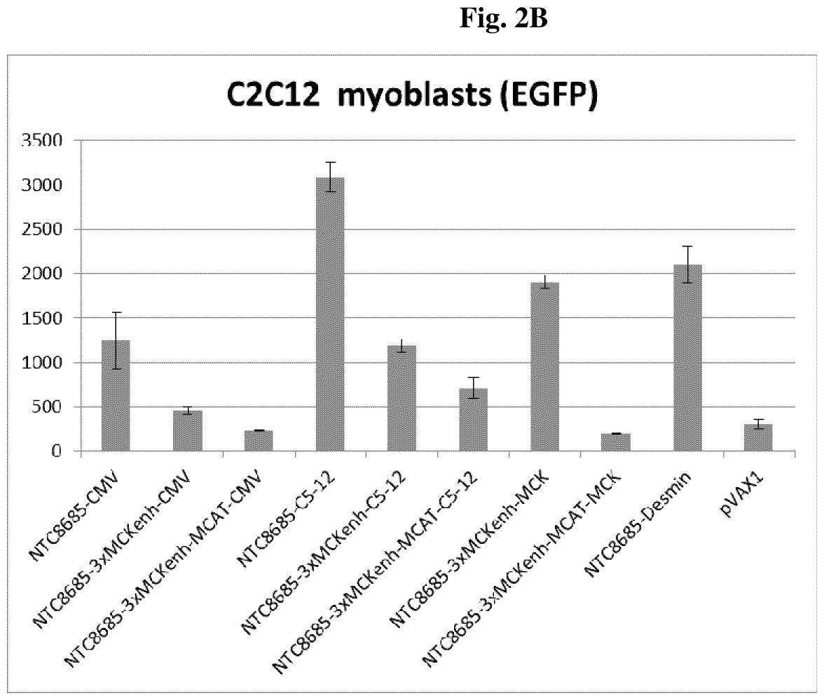

The resulting EGFP expression levels for HEK293 cells and C5-C12 myoblasts are shown in FIGS. 2A-2B.

As shown in FIGS. 2A-2B, the C5-12 randomly assembled synthetic promoter (Li X, Eastman E M, Schwartz R J, Draghia-Akli R D 1999. Nature Biotech 17:241), was the strongest muscle promoter, followed by hDesmin then mMCK (combining 3 copies of the CK7 enhancer SEQ ID NO: 1 with the murine MCK promoter −357-+7 SEQ ID NO:40). Interestingly attempts to create hybrid promoters with improved activity by modification of the CMV, C5-12 and MCK promoters to add muscle specific enhancer elements instead reduced the promoter strength. Addition of 3 copies of the CK7 MCK enhancer of SEQ ID NO: 1 upstream of the CMV or C5-12 promoters dramatically reduced muscle expression. Similarly addition of 2 copies (SEQ ID NO: 47) of an M-CAT (muscle-CAT motif that contains 5′CATTCCT-3′ TEF-1 binding sites; Li et al, Supra, 1999) (SEQ ID NO: 46) between the CK7 MCK enhancer of SEQ ID NO: 1 and the C5-12 and MCK promoters further reduced muscle specific expression. This is consistent with the teachings of Souza and Armentano, WO2002095006 who obtained relatively weak promoters by combining an MCK enhancer with the hDesmin promoter. Collectively, these results suggest that the CK7 MCK enhancer and the M-CAT motif decrease muscle specific expression when cloned upstream of a heterologous promoter. Consistent with this, all the strong promoters in Table 1 that use MCK enhancers incorporate them upstream of the MCK promoter.

Other promoter-enhancer combinations such as tMCK (Wang B, Li J, Fu F H, Chen C, Zhu X, Zhou L, Jiang X, Xiao X. 2008. Gene Ther 15:1489) and Sk-CRM4-Des (Sarcar S, Tualamba W, et al. 2019. Nat Comm 10:492) were shown to create hybrid muscle promoters with activities exceeding the CMV promoter (Table 1). These promoters were tested using linear AAV vectors and may not function similarly in supercoiled plasmid or Nanoplasmid DNA templates. To test this, several muscle promoter expression vectors were created, in pVAX1 (pUC origin kanR vector; Invitrogen) and the NTC9385R Nanoplasmid backbones. This allowed evaluation of impact of the 1) muscle promoter per se; and 2) vector backbone on muscle cell expression. Expression results are shown in Table 3. All vectors have the same Bovine Growth Hormone derived polyadenylation signal.

TABLE 3
EGFP Expression for Various Constructs

| EGFP | | | | | | |
|---|---|---|---|---|---|---|
| Plasmid | First | Second | | | | |
| (CRM = | Enhancer | Enhancer | | | C2C12 | HEK |
| SK-SH4) | Source | Source | Promoter | Intron | EGFP | EGFP |
| pVAX1 | | CMV | CMV | None | 70 ± 4 | 2542 ± |
| (CMV) | | (SEQ ID NO: | SEQ ID | | | 683 |
| | | 51) | NO: 51) | | | |
| NTC9385R | | CMV | CMV | HR-β | 1348 ± | 36516 ± |
| (CMV) | | (SEQ ID NO: | (SEQ ID | (SEQ ID | 122 | 2891 |
| | | 45) | NO: 45) | NO: 53) | | |
| NTC9385R- | SK-CRM4 | CMV | CMV | HR-β | 993 ± | 31793 ± |
| CRM-CMV- | (myosin light | (SEQ ID NO: | (SEQ ID | (SEQ ID | 97 | 1958 |
| BGH pA | chain | 45) | NO: 45) | NO: 53) | | |
| | enhancer) | | | | | |
| | (SEQ ID | | | | | |
| | NO: 14 | | | | | |
| pVAX1- | SK-CRM4 | mMCK 2R5S | mMCK −80-+7 | MVM | 45 ± 2 | 38 ± 2 |
| CRM- | (myosin light | (3x) | (SEQ ID | intron | | |
| tMCK- | chain | (SEQ ID NO: | NO: 39) | (SEQ ID | | |
| intron = | enhancer) | 37 (3x)) | | NO: 17) | | |
| Sk- | (SEQ ID | | | | | |
| CRM4- | NO: 14) | | | | | |
| tMCK | | | | | | |
| promoter | | | | | | |
| NTC9385R- | SK-CRM4 | mMCK 2R5S | mMCK −80-+7 | MVM | 148 ± | 46 ± 2 |
| CRM- | (myosin light | (3x) | (SEQ ID | intron | 44 | |
| tMCK- | chain | (SEQ ID NO: | NO: 39) | (SEQ ID | | |
| intron = | enhancer) | 37 (3x)) | | NO: 17) | | |
| Sk- | (SEQ ID | | | | | |
| CRM4- | NO: 14) | | | | | |
| tMCK | | | | | | |
| promoter | | | | | | |
| NTC9385R- | NA | mMCK 2R5S | mMCK −80-+7 | MVM | 350 ± | 55 ± 2 |
| tMCK- | | (3x) | (SEQ ID | intron | 41 | |
| intron = | | (SEQ ID NO: | NO: 39) | (SEQ ID | | |
| tMCK | | 37 (3x)) | | NO: 17) | | |
| promoter | | | | | | |
| NTC9385R- | NA | mMCK 2R5S | mMCK −80-+7 | MVM | 227 ± 7 | 43 ± 2 |
| tMCK- | | (3x) | (SEQ ID | MCK- | | |
| intron (SIE) | | (SEQ ID NO: | NO: 39) | SIE | | |
| | | 37 (3x)) | | intron | | |
| | | | | (SEQ ID | | |
| | | | | NO: 42) | | |
| NTC9385R- | NA | mMCK 2R5S | mMCK −80-+7 | MVM | 469 ± | 145 ± |
| tMCK-INR- | | (3x) | INR | MCK- | 53 | 38 |
| intron(SIE) | | (SEQ ID NO: | (SEQ ID | SIE | | |
| | | 37 (3x)) | NO: 43) | intron | | |
| | | | | (SEQ ID | | |
| | | | | NO: 42) | | |
| NTC9385R- | NA | mMCK CK7 | mMCK −357-+7 | HR-β | 1351 ± | 111 ± 5 |
| 3xMCKenh- | | (−1262-−1060) | (SEQ ID | (SEQ ID | 153 | |
| MCK | | (SEQ | NO: 40) | NO: 53) | | |
| | | ID NO: | | | | |
| | | 1(3x)) | | | | |
| pVAX1- | SK-CRM4 | mDesmin | mDesmin | MVM | 246 ± | 43 ± 5 |
| CRM- | (myosin light | (−895-−592) | (−591-+83) | intron | 13 | |
| mDesmin- | chain | (SEQ ID NO: | (SEQ ID | (SEQ ID | | |
| BGH pA = | enhancer) | 3) | NO: 4) | NO: 17) | | |
| Sk- | (SEQ ID NO: | | | | | |
| CRM4-Des | 14) | | | | | |
| promoter | | | | | | |
| NTC9385R- | SK-CRM4 | mDesmin | mDesmin | MVM | 2582 ± | 94 ± 2 |
| CRM- | (myosin light | (−895-−592) | (−591-+83) | intron | 98 | |
| mDesmin = | chain | (SEQ ID NO: | (SEQ ID | (SEQ ID | | |
| Sk- | enhancer) | 3) | NO: 4) | NO: 17) | | |
| CRM4-Des | (SEQ ID NO: | | | | | |
| promoter | 14) | | | | | |
| NTC9385R- | SK-CRM4 | mDesmin | mDesmin | MVM | 1425 ± | 68 ± 3 |
| CRM- | (myosin light | (−895-−592) | (−591-+83) | MCK- | 103 | |
| mDesmin | chain | (SEQ ID NO: | (SEQ ID | SIE | | |
| (SIE) | enhancer) | 3) | NO: 4) | intron | | |
| | (SEQ ID NO: | | | (SEQ ID | | |
| | 14) | | | NO: 42) | | |
| NTC9385R- | mMCK | mDesmin | mDesmin | MVM | 1144 ± | 69 ± 7 |
| tMCKE- | 2R5S (3x) | (−895-−592) | (−591-+83) | intron | 85 | |
| mDesmin | (SEQ ID | (SEQ ID NO: | (SEQ ID | (SEQ ID | | |
| | NO: 37 (3x)) | 3) | NO: 4) | NO: 17) | | |
| NTC9385R- | mMCK | mDesmin | mDesmin | MVM | 958 ± | 62 ± 1 |
| tMCKE- | 2R5S (3x) | (−895-−592) | (−591-+83) | MCK- | 38 | |
| mDesmin | (SEQ ID | (SEQ ID NO: | (SEQ ID | SIE | | |
| (SIE) | NO: 37 (3x)) | 3) | NO: 4) | intron | | |
| | | | | (SEQ ID | | |
| | | | | NO: 42) | | |

The vector backbone had a dramatic effect on expression. pVAX1 expression was 10-20-fold lower than NTC9385R with CMV, and the Sk-CRM4-Des promoter, and 3-fold lower with a new hybrid Sk-CRM4-tMCK promoter. This teaches that the Nanoplasmid backbone dramatically improves expression compared to pVAX1.

Using pVAX1 CMV as a baseline for CMV promoter expression, all the muscle specific Nanoplasmid vectors are improved expression in muscle cells compared to CMV.

However, compared to Nanoplasmid CMV as a baseline, only the Sk-CRM4-Des promoter is improved compared to CMV.

The tMCK promoter expression was much lower than Sk-CRM4-Des promoter. As observed in FIGS. 2A-2B with the CK7 MCK enhancer and the M-CAT motif, addition of a muscle enhancer to the tMCK promoter (intronic MCK SIE enhancer SEQ ID NO: 41; Tai P W L, Fisher-Aylor K I, Himeda C L, Smith C L, MacKenzie A P, Helterline D L, Agnello J C, Welikson R E<Wold B H, Hauschka S D. 2011. Skeletal Muscle 1:25) decreased expression (Table 3). The intronic MCK SIE enhancer also 2 fold reduced expression from the Sk-CRM4-Des promoter, and slightly reduced expression from the tMCKE-mDesmin promoter.

The mMCK 2RS5 enhancer SEQ ID NO: 38 (3 copies of SEQ ID NO: 37; Wang et al, Supra, 2008) was tested in combination with mDesmin enhancer and promoter (tMCKE-mDesmin), to determine if a MCK enhancer could substitute for Sk-CRM4 in the Sk-CRM4-Des promoter. However, NTC9385R-tMCKE-mDesmin was 2-fold lower activity in muscle cells than NTC9385R-CRM-mDesmin.

Collectively these data suggest that MCK enhancers may not improve expression in muscle cells when cloned upstream of CMV, C5-12 and desmin promoters and that most combinations of muscle specific promoter elements are detrimental rather than beneficial to expression levels from these promoters.

### Example 2: Evaluation of Novel MCK Desmin Muscle Promoters in pVAX1 and Nanoplasmid Backbones

Of the tested promoters from Example 1, the Sk-CRM4-Des promoter was strongest for expression in the Nanoplasmid vector backbone. Surprisingly, contrary to the results above, replacement of the Sk-CRM4-Des promoter in NTC9385R-CRM-mDesmin with 1 or 3 copies of the MCK CK7 enhancer (SEQ ID NO: 1) created novel NTC9385R-MCK CK7 E vectors with muscle specific expression equivalent to the Sk-CRM4-Des promoter as shown in Table 4 below. The same methodologies as used in Example 1 were to used to generate this additional data. All vectors have the same Bovine Growth Hormone derived polyadenylation signal.

TABLE 4
EGFP Expression for Various Constructs

| EGFP | | | | | | | |
|---|---|---|---|---|---|---|---|
| Plasmid | First | Second | | | Fold | Fold | Fold |
| (CRM = | Enhancer | Enhancer | | | pVAX1 | pVAX1 | pVAX1 |
| SK-SH4) | source | source | Promoter | Intron | (Exp1) | (Exp1) | (Exp1) |
| pVAX1 | | CMV | CMV | None | 1x | 1x | 1x |
| (CMV) | | (SEQ ID | (SEQ ID | | | | |
| | | NO: 51) | NO: 51) | | | | |
| NTC9385R | | CMV | CMV | HR-β | 6.9x | 6.2x | |
| (CMV) | | (SEQ ID | (SEQ ID | (SEQ ID | | | |
| | | NO: 45) | NO: 45) | NO: 53) | | | |
| pVAX1- | SK-CRM4 | mDesmin | mDesmin | MVM | 1.3x | 0.9x | 1.1x |
| CRM- | (myosin | (−895-−592) | (−591-+83) | intron | | | |
| mDesmin | light chain | (SEQ ID | (SEQ ID | (SEQ ID | | | |
| | enhancer) | NO: 3) | NO: 4) | NO: 17) | | | |
| | (SEQ ID | | | | | | |
| | NO: 14) | | | | | | |
| NTC9385R- | SK-CRM4 | mDesmin | mDesmin | MVM | 11.9x | 4.3x | 3.4x |
| CRM- | (myosin | (−895-−592) | (−591-+83) | intron | | | |
| mDesmin | light chain | (SEQ ID | (SEQ ID | (SEQ ID | | | |
| | enhancer) | NO: 3) | NO: 4) | NO: 17) | | | |
| | (SEQ ID | | | | | | |
| | NO: 14) | | | | | | |
| NTC9385R- | mMCK | mDesmin | mDesmin | MVM | 9.3x | 5.0x | 2.6x |
| MCK 3x | CK7 | (−895-−592) | INR | intron | | | |
| CK7 E | (−1262-−1060) | (SEQ ID | (−591-+83) | (SEQ ID | | | |
| mDesmin- | (SEQ ID | NO: 3) | (SEQ ID | NO: 17) | | | |
| INR | NO: 1(3x)) | | NO: 5) | | | | |
| | 3x = SEQ | | | | | | |
| | ID NO: 2 | | | | | | |
| NTC9385R- | mMCK | mDesmin | mDesmin | MVM | 11.3x | 5.6x | 2.6x |
| MCK 1x | CK7 | (−895-−592) | INR | intron | | | |
| CK7 E | (−1262-−1060) | (SEQ ID | (−591-+83) | (SEQ ID | | | |
| mDesmin- | (SEQ ID | NO: 3) | (SEQ ID | NO: 17) | | | |
| INR | NO: 1) | | NO: 5) | | | | |
| NTC9385R- | mMCK | mDesmin | mDesmin | MVM | 12.5x | 4.6x | 3.9x |
| MCK 3x | CK7 | (−895-−592) | (−591-+83) | intron | | | |
| CK7 E | (−1262-−1060) | (SEQ ID | (SEQ ID | (SEQ ID | | | |
| mDesmin | (SEQ ID | NO: 3) | NO: 4) | NO: 17) | | | |
| | NO: 1(3x)) | | | | | | |
| | 3x = SEQ | | | | | | |
| | ID NO: 2 | | | | | | |
| NTC9385R- | mMCK | mDesmin | mDesmin | MVM | 9.6x | 4.1x | 2.9x |
| MCK 1x | CK7 | (−895-−592) | (−591-+83) | intron | | | |
| CK7 E | (−1262-−1060) | (SEQ ID | (SEQ ID | (SEQ ID | | | |
| mDesmin | (SEQ ID | NO: 3) | NO: 4) | NO: 17) | | | |
| | NO: 1) | | | | | | |
| NTC9385R- | NA | mMCK | mMCK −357-+7 | HR-β | 7.4x | 3.3x | 2.0x |
| 3xMCKenh- | | CK7 | (SEQ ID | (SEQ ID | | | |
| MCK | | (−1262-−1060) | NO: 40) | NO: 53) | | | |
| | | (SEQ ID | | | | | |
| | | NO: | | | | | |
| | | 1(3x)) | | | | | |

The NTC9385R-MCK CK7 E vectors have hybrid muscle promoters comprising:
a) 1 to 3 copies of the MCK CK7 enhancer 5′ to:
b) The mDesmin enhancer which is 5′ to:
c) The mDesmin promoter (with or without INR) which is 5′ to:
d) The MVM intron.

The mDesmin INR promoter was constructed similarly to the MCK INR disclosed in Salva et al 2007. Mol Ther 15:320. The INR (initiator—a core promoter element) changes increase the activity of the TATA box to increase transcriptional initiation. The INR change slightly increases promoter expression in non muscle cells (Table 5; HEK293 and A549 cells) but the INR containing promoters remain highly specific to muscle cells.

The EGFP expression levels for various constructs are provided in Table 5 below. All vectors have the same Bovine Growth Hormone derived polyadenylation signal. Enhancer, promoter and intron sequences are as described in Table 4.

TABLE 5
EGFP Expression for Various Constructs

| EGFP | | | | | | | |
|---|---|---|---|---|---|---|---|
| Plasmid | | | | | | | |
| (CRM = SK- | | | | | C2C12 | HEK | A549 |
| SH4) | Backbone | Enhancers | Promoter | Intron | EGFP | EGFP | EGFP |
| pVAX1 | pUC-KanR | CMV | CMV | None | 94 ± 24 | 2632 ± | 489 ± |
| (CMV) | | | | | | 328 | 25 |
| pVAX1- | pUC-KanR | SK-SH4- + | mDesmin | MVM | 87 ± 15 | 39 ± 2 | 37 ± 4 |
| CRM- | | mDesmin | | | | | |
| mDesmin- | | | | | | | |
| NTC9385R- | R6K-RNA- | SK-SH4- + | mDesmin | MVM | 398 ± 6 | 52 ± 3 | 49 ± 3 |
| CRM- | OUT | mDesmin | | | | | |
| mDesmin- | (SEQ ID | | | | | | |
| | NO: 27) | | | | | | |
| NTC9385R- | R6K-RNA- | 3x CK7 E + | mDesmin | MVM | 429 ± | 58 ± 1 | 48 ± 5 |
| MCK 3x CK7 | OUT | mDesmin | | | 35 | | |
| E mDesmin | (SEQ ID | | | | | | |
| | NO: 27) | | | | | | |
| NTC9385R- | R6K-RNA- | 3x CK7 E + | mDesmin- | MVM | 473 ± | 330 ± | 112 ± |
| MCK 3x CK7 | OUT | mDesmin | INR | | 25 | 47 | 8 |
| E mDesmin- | (SEQ ID | | | | | | |
| INR | NO: 27) | | | | | | |

Substitution of 3 copies of the MCK CK7 enhancer with 3 copies of the MCK2R enhancer two-fold reduced expression (Table 6: NTC9385R-MCK 3×MCK2R mDesmin-pCI versus NTC9385R-MCK 3×CK7 E mDesmin-pCI). This suggests that the 2R modification (Hauser et al 2000. Mol Ther 2: 16) that changes the left E-Box to match the right E-box is detrimental to expression in this context with the Desmin promoter.

NTC9385R Nanoplasmid MCK-Desmin promoter vectors were constructed in which the murine Desmin enhancer promoter was substituted with the human Desmin enhancer promoter (with and without INR and short and long versions; the short versions remove the negative region within the promoter reported in Li, Z and Paulin D. 1991. J Biol Chem 266:6562). As well, MVM intron and pCI intron versions of both MCK mDesmin and MCK hDesmin promoters were constructed and tested for expression in muscle cells. The results are provided in Tables 6 and 7.

TABLE 6
EGFP Expression for Various Constructs

| | | | | | C2C12 | C2C12 |
|---|---|---|---|---|---|---|
| | First | Second | | | T = 6 | T = 6 |
| | Enhancer | Enhancer | | | FU | FU |
| EGFP Plasmid ^{a} | source | source | Promoter | Intron | Exp 1 | Exp 2 |
| pVAX1 (CMV) | | CMV | CMV | None | 3.0 ± 0.2 | 3.2 ± |
| | | (SEQ ID | (SEQ ID | | | 0.2 |
| | | NO: 51) | NO: 51) | | | |
| NTC9385R | | CMV | CMV | HR-β | 72.7 ± | 38.1 ± |
| (CMV) | | (SEQ ID | (SEQ ID | (SEQ ID | 9.6 | 11.2 |
| | | NO: 45) | NO: 45) | NO: 53) | | |
| NTC9385R- | mMCK | mDesmin | mDesminINR | MVM | 172.9 ± | 97.5 ± |
| MCK 3x CK7 E | CK7 | (−895-−592) | (−591-+83) | intron | 27.6 | 9.4 |
| mDesmin-INR | (−1262-−1060) | (SEQ ID | (SEQ ID | (SEQ ID | | |
| | (SEQ ID | NO: 3) | NO: 5) | NO: 17) | | |
| | NO: 1(3x)) | | | | | |
| | 3x = SEQ | | | | | |
| | ID NO: 2 | | | | | |
| NTC9385R- | mMCK | mDesmin | mDesmin | MVM | 123.5 ± | 51.8 ± |
| MCK 3x CK7 E | CK7 | (−895-−592) | (−591-+83) | intron | 29.1 | 15.4 |
| mDesmin | (−1262-−1060) | (SEQ ID | (SEQ ID | (SEQ ID | | |
| | (SEQ ID | NO: 3) | NO: 4) | NO: 17) | | |
| | NO: 1(3x)) | | | | | |
| | 3x = SEQ | | | | | |
| | ID NO: 2 | | | | | |
| NTC9385R- | mMCK | mDesmin | mDesmin | pCI | 207.1 ± | 78.8 ± |
| MCK 3x CK7 E | CK7 | (−895-−592) | (−591-+83) | intron | 44.2 | 7.5 |
| mDesmin-pCI | (−1262-−1060) | (SEQ ID | (SEQ ID | (SEQ ID | | |
| | (SEQ ID | NO: 3) | NO: 4) | NO: 18) | | |
| | NO: 1(3x)) | | | | | |
| | 3x = SEQ | | | | | |
| | ID NO: 2 | | | | | |
| NTC9385R- | mMCK | mDesmin | mDesmin | pCI | 98.3 ± | 49.4 ± |
| MCK 3x | MCK2R | (−895-−592) | (−591-+83) | intron | 8.1 | 4.7 |
| MCK2R | (−1262-−1060) | (SEQ ID | (SEQ ID | (SEQ ID | | |
| mDesmin-pCI | (SEQ ID | NO: 3) | NO: 4) | NO: 18) | | |
| | NO: 54 | | | | | |
| | (3x)) | | | | | |
| NTC9385R- 3x | mMCK | hDesmin) | hDesminP | MVM | 139.2 ± | 70.4 ± |
| MCK CK7 E | CK7 | (−990-−620) | (−619-+86) | intron | 16.7 | 14.3 |
| hDesmin | (−1262-−1060) | (SEQ ID | (SEQ ID | (SEQ ID | | |
| | (SEQ ID | NO: 6) | NO: 7) | NO: 17) | | |
| | NO: 1(3x)) | | | | | |
| | 3x = SEQ | | | | | |
| | ID NO: 2 | | | | | |
| NTC9385R- 3x | mMCK | hDesmin) | hDesminINRP | MVM | 309.3 ± | 363.4 ± |
| MCK CK7 E | CK7 | (−990-−620) | (−619-+86) | intron | 27.7 | 396 |
| hDesmin-INR | (−1262-−1060) | (SEQ ID | (SEQ ID | (SEQ ID | | (high |
| | (SEQ ID | NO: 6) | NO: 8) | NO: 17) | | SD) |
| | NO: 1(3x)) | | | | | |
| | 3x = SEQ | | | | | |
| | ID NO: 2 | | | | | |
| NTC9385R- 3x | mMCK | hDesmin) | hDesminSP | MVM | 179.2 ± | 81.2 ± |
| MCK CK7 E | CK7 | (−990-−620) | (−619-+86) | intron | 18.3 | 8.3 |
| hDesminS | (−1262-−1060) | (SEQ ID | (SEQ ID | (SEQ ID | | |
| | (SEQ ID | NO: 6) | NO: 9) | NO: 17) | | |
| | NO: 1(3x)) | | | | | |
| | 3x = SEQ | | | | | |
| | ID NO: 2 | | | | | |
| NTC9385R- 3x | mMCK | hDesmin) | hDesminINR | MVM | 285.3 ± | 140.7 ± |
| MCK CK7 E | CK7 | (−990-−620) | SP | intron | 30.7 | 25.6 |
| hDesminS-INR | (−1262-−1060) | (SEQ ID | (−619-+86) | (SEQ ID | | |
| | (SEQ ID | NO: 6) | (SEQ ID | NO: 17) | | |
| | NO: 1(3x)) | | NO: 10) | | | |
| | 3x = SEQ | | | | | |
| | ID NO: 2 | | | | | |

^{a} pVAX1 bacterial backbone has 148 CpG motifs. NTC9385R backbone has 20 CpG motifs

TABLE 7
EGFP Expression for Various Constructs

| | | | | | C2C12 | C2C12 |
|---|---|---|---|---|---|---|
| | First | Second | | | T = 6 | T = 6 |
| | Enhancer | Enhancer | | | FU | FU |
| EGFP Plasmid ^{a} | source | source | Promoter | Intron | Exp 1 | Exp 2 |
| pVAX1 (CMV) | CMV | CMV | CMV | None | 8.4 ± | 15.1 ± |
| | | | | | 1.1 | 1.3 |
| NTC9385R- | mMCK | mDesmin | mDesminINR | MVM | 38.7 ± | 174.7 ± |
| MCK 3x CK7 E | CK7 | (−895-−592) | (−591-+83) | intron | 10.8 | 42.8 |
| mDesmin-INR | (−1262-−1060) | (SEQ ID | (SEQ ID | (SEQ | | |
| | (SEQ | NO: 3) | NO: 5) | ID NO: | | |
| | ID NO: | | | 17) | | |
| | 1(3x)) 3x = | | | | | |
| | SEQ ID | | | | | |
| | NO: 2 | | | | | |
| NTC9385R- | mMCK | mDesmin | mDesmin | PCI | 47.7 ± | 154.9 ± |
| MCK 3x CK7 E | CK7 | (−895-−592) | (−591-+83) | intron | 14.2 | 32.2 |
| mDesmin-pCI | (−1262-−1060) | (SEQ ID | (SEQ ID | (SEQ | | |
| | (SEQ | NO: 3) | NO: 4) | ID NO: | | |
| | ID NO: | | | 18) | | |
| | 1(3x)) 3x = | | | | | |
| | SEQ ID | | | | | |
| | NO: 2 | | | | | |
| NTC9385R- 3x | mMCK | hDesmin) | hDesminINR P | MVM | 51.6 ± | 176.1 ± |
| MCK CK7 E | CK7 | (−990-−620) | (−619-+86) | intron | 8.4 | 23 |
| hDesmin-INR | (−1262-−1060) | (SEQ ID | (SEQ ID | (SEQ | | |
| | (SEQ | NO: 6) | NO: 8) | ID NO: | | |
| | ID NO: | | | 17) | | |
| | 1(3x)) 3x = | | | | | |
| | SEQ ID | | | | | |
| | NO: 2 | | | | | |
| NTC9385R- 3x | mMCK | hDesmin) | hDesminINR P | pCI | 90.0 ± | 278.0 ± |
| MCK CK7 E | CK7 | (−990-−620) | (−619-+86) | intron | 12.9 | 54.2 |
| hDesmin-INR | (−1262-−1060) | (SEQ ID | (SEQ ID | (SEQ | | |
| pCI | (SEQ | NO: 6) | NO: 8) | ID NO: | | |
| | ID NO: | | | 18) | | |
| | 1(3x)) 3x = | | | | | |
| | SEQ ID | | | | | |
| | NO: 2 | | | | | |
| NTC9385R- 3x | mMCK | hDesmin) | hDesminSP | MVM | 47.5 ± | 98.8 ± |
| MCK CK7 E | CK7 | (−990-−620) | (−619-+86) | intron | 7.7 | 24.5 |
| hDesminS | (−1262-−1060) | (SEQ ID | (SEQ ID | (SEQ | | |
| | (SEQ | NO: 6) | NO: 9) | ID NO: | | |
| | ID NO: | | | 17) | | |
| | 1(3x)) 3x = | | | | | |
| | SEQ ID | | | | | |
| | NO: 2 | | | | | |
| NTC9385R- 3x | mMCK | hDesmin) | hDesminINR SP | MVM | 61.8 ± | 170.4 ± |
| MCK CK7 E | CK7 | (−990-−620) | (−619-+86) | intron | 16.6 | 26.8 |
| hDesminS-INR | (−1262-−1060) (SEQ | (SEQ ID | (SEQ ID | (SEQ | | |
| | ID NO: | NO: 6) | NO: 10) | ID NO: | | |
| | 1(3x)) 3x = | | | 17) | | |
| | SEQ ID | | | | | |
| | NO: 2 | | | | | |
| NTC9385R- 3x | mMCK | hDesmin) | hDesminINR SP | PCI | 72.8 ± | 270.8 ± |
| MCK CK7 E | CK7 | (−990-−620) | (−619-+86) | intron | 22.7 | 28.5 |
| hDesminS-INR | (−1262-−1060) | (SEQ ID | (SEQ ID | (SEQ ID | | |
| pCI | (SEQ | NO: 6) | NO: 10) | NO: 18) | | |
| | ID NO: | | | | | |
| | 1(3x)) 3x = | | | | | |
| | SEQ ID | | | | | |
| | NO: 2 | | | | | |

^{a} pVAX1 bacterial backbone has 148 CpG motifs. NTC9385R backbone has 20 CpG motifs

The data demonstrate that MCK hDesmin is better than MCK mDesmin for expression in myotubes; MCK hDesminS and MCK hDesmin have similarly high expression in myotubes; including INR provides an improvement in expression in myotubes; the pCI intron demonstrates improved expression than the MVM intron in myotubes; and Nanoplasmid vectors show significant improvement in expression over the pVAX1 vector in myotubes.

The muscle-specific regulatory nucleic acid sequences and vectors of the present disclosure can also have a favorably low CpG to GpG ratio (Table 8). Lower CpG to GpG ratio correlates with reduced transgene immunogenicity, hypothesized through GpG competition with CpG for TLR9 binding (Gottlieb P, Utz P J, Robinson W, Steinman L. 2013. Clin Immunol 149:297). Nanoplasmid vectors incorporating muscle promoters of the current invention also have a favorably low CpG to GpG ratio, especially compared to existing CMV promoter vectors such as pVAX1 (Table 9). This could lead to reduced immune response against target transgenes which would be highly beneficial for gene therapy and passive immunotherapy applications where immune responses are a problem (Weeratna et al, Supra, 2001; Hollevoet and Declerck Supra, 2017).

TABLE 8
Promoter Characteristics and CpG to GpG Ratio

| | | | | | CpG | GpG | |
|---|---|---|---|---|---|---|---|
| | | | | | motifs | motifs | CpG/ |
| | | Promoter | | Expression | (per | (per | GpG |
| Vector name | Promoter | size | Intron | level | kb) | kb) | ratio ^{a} |
| pVAX1 | CMV | 726 bp | No | Low | 39 | 50 | 0.78 |
| CMV | | | | | (54) | (69) | |
| NTC9385R | CMV | 1063 bp | HR β | High | 70 | 64 | 1.09 |
| CMV | | | | | (66) | (60) | |
| NTC9385R | MCK 3x | 1607 bp | MVM | High | 55 | 183 | 0.30 |
| MCK | CK7 E | | | (muscle | (34) | (114) | |
| mDesmin | mDesmin- | | | specific) | | | |
| muscle | INR | | | | | | |
| promoter | | | | | | | |
| NTC9385R | MCK 3x | 1717 bp | PCI | High | 53 | 195 | 0.27 |
| MCK | CK7 E | | | (muscle | (31) | (112) | |
| hDesmin | hDesmin- | | | specific) | | | |
| muscle | INR | | | | | | |
| promoter | | | | | | | |
| SEQ ID | | | | | | | |
| NO: 35 | | | | | | | |
| NTC9385R | MCK 3x | 1244 bp | pCI | High | 43 | 150 | 0.29 |
| MCK | CK7 E | | | (muscle | (35) | (121) | |
| hDesminS | hDesminS- | | | specific) | | | |
| muscle | INR | | | | | | |
| promoter | | | | | | | |
| SEQ ID | | | | | | | |
| NO: 36 | | | | | | | |
| CAG | CAG | 1764 bp | Chicken | High | 190 | 273 | 0.70 |
| promoter | | | B globin | | (108) | (155) | |

^{a} Lower CpG to GpG ratio correlates with reduced transgene immunogenicity, hypothesized through GpG competition with CpG for TLR9 binding (Gottlieb et al, Supra. 2013).

TABLE 9
Plasmid and Nanoplasmid Vector CpG Characteristics

| | | | | | | CpG | GpG | |
|---|---|---|---|---|---|---|---|---|
| | | | | Prom- | Trans- | motifs | motifs | CpG/ |
| Vector | Vector | Backbone | | oter | gene | (per | (per | GpG |
| name^{a} | size | (CpG) | Promoter | (CpG) | (CpG) | kb) | kb) | ratio ^{a} |
| pVAX1- | 3685 | KanR- | CMV | 39 | 60 | 249 | 288 | 0.87 |
| EGFP | bp | pUC | | | | (68) | (78) | |
| | | (145 | | | | | | |
| | | CpG) | | | | | | |
| NTC9385R- | 2487 | R6K- | CMV- | 70 | 60 | 151 | 159 | 0.95 |
| EGFP | bp | ROUT | HTLV-IR | | | (61) | (64) | |
| | | (16 CpG) | | | | | | |
| NTC9385R- | 3094 | R6K- | CAG | 190 | 60 | 268 | 363 | 0.74 |
| CAG | bp | ROUT | | | | (87) | (117) | |
| EGFP | | (16 CpG) | | | | | | |
| NTC9385R | 3028 | R6K- | 3xMCK- | 55 | 60 | 132 | 279 | 0.48 |
| MCK- | bp | ROUT | mDesmin- | | | (44) | (92) | |
| mDesmin | | (16 CpG) | MVM | | | | | |
| EGFP | | | | | | | | |
| NTC9385R- | 3073 | R6K- | 3xMCK- | 53 | 60 | 132 | 268 | 0.49 |
| MCK- | bp | ROUT | hDesmin | | | (43) | (87) | |
| hDesmin | | (16 CpG) | INR-pCI | | | | | |
| EGFP | | (SEQ ID | (SEQ ID | | | | | |
| | | NO: 27 & | NO: 35) | | | | | |
| | | 29) | | | | | | |
| NTC9385R | 3155 | 1x CpG | 3xMCK- | 53 | 60 | 119 | 289 | 0.41 |
| (3xCpG)- | bp | R6K; 2x | hDesmin | | | (38) | (92) | |
| MCK- | | CpG | INR-pCI | | | | | |
| hDesmin | | ROUT | (SEQ ID | | | | | |
| EGFP | | (3 CpG) | NO: 35) | | | | | |
| | | (SEQ ID | | | | | | |
| | | NO: 28 & | | | | | | |
| | | 30) | | | | | | |
| NTC9385R- | 2601 | R6K- | 3xMCK- | 43 | 60 | 124 | 225 | 0.55 |
| MCK- | | ROUT | hDesminS | | | (48) | (87) | |
| hDesminS | | (16 CpG) | INR-pCI | | | | | |
| EGFP | | (SEQ ID | (SEQ ID | | | | | |
| | | NO: 27 & | NO: 36) | | | | | |
| | | 29) | | | | | | |
| NTC9385R | 2683 | 1x CpG | 3xMCK- | 43 | 60 | 111 | 246 | 0.45 |
| (3xCpG)- | bp | R6K; 2x | hDesminS | | | (41) | (92) | |
| MCK- | | CpG | INR-pCI | | | | | |
| hDesminS | | ROUT | (SEQ ID | | | | | |
| EGFP | | (3 CpG) | NO: 36) | | | | | |
| | | (SEQ ID | | | | | | |
| | | NO: 28 & | | | | | | |
| | | 30) | | | | | | |

^{a} Lower CpG to GpG ratio correlates with reduced transgene immunogenicity, hypothesized through GpG competition with CpG for TLR9 binding (Gottlieb et al, Supra. 2013). Reduction of the CpG/GpG ratio from 0.71 (pVAX1-INS) to 0.65 (BHT-3021-INS; modified pVAX1) resulted in improved insulin tolerizing DNA vaccine performance (Solvason N, Lou Y-P, Peters W, Evans E, Martinez J, Ramirez U, Ocampo A, Yun R, Ahmad S, Liu E, Yu L, Eisenbarth G, Leviten M, Steinman L, Garren H. 2008. J Immunol 181: 8298).

### Example 3: In Vivo Evaluation of Novel MCK Desmin Muscle Promoters in a Nanoplasmid Vector Backbone

The MCK Desmin muscle promoter Nanoplasmid vectors can be evaluated in vivo for improved expression compared to pVAX1 CMV control. For example, luciferase transgene versions of pVAX1 and the FIGS. 3A-3D Nanoplasmid MCK-hDesmin vectors and FIGS. 5A-5D Nanoplasmid MCK-mDesmin vectors will be created, and purified DNA delivered in vivo to mouse muscle. Delivery will be by IM delivery with electroporation, TI delivery with block polymers, such as X-shaped Poloxamines=Tetronic 304, 704, 904, 908 from BASF (Ludwigshafen, Germany), IM delivery with lipids, etc. Luciferase expression will be determined in injected muscles at various timepoints. Nanoplasmid MCK Desmin muscle promoter expression levels 1-2 logs superior to pVAX1 CMV are expected.

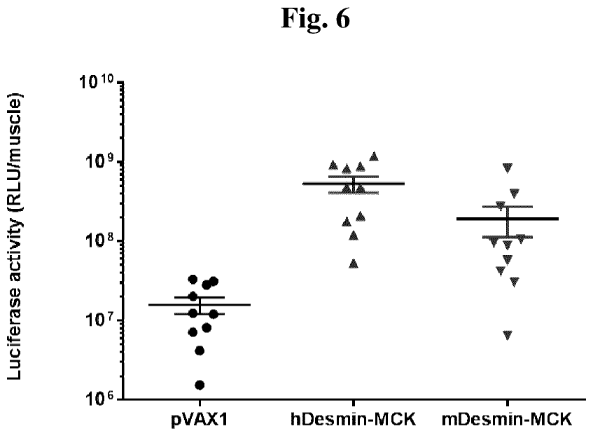

For example, block polymer delivery of luciferase transgene versions of FIG. 3C MCK-hDesmin Nanoplasmid [NTC9385R (3×CpG)-MCK hDesmin-Luc CpG free BGH pA (4099 bp)] and FIG. 5B MCK-mDesmin Nanoplasmid [NTC9385R (3×CpG)-MCK mDesmin-Luc CpG free BGH pA (3948 bp)] in vivo to mouse muscle demonstrated >1 log improved expression compared to pVAX1 plasmid [pVAX1-Luc (4613 bp)] as described below (FIG. 6).

Formulations of the 3 DNA preparations with Amphiphilic Block Copolymer (ABC) (Nanotaxi; In-Cell-Art, Nantes, France) were prepared by mixing equal volumes of ABC stock solution in water and plasmid DNA solution at the desired concentration in buffered solution.

Animal experiments were performed according to institutional and national ethical guidelines. Mice were anesthetized by isoflurane before injection of ABC/DNA solution. Mouse luciferase gene expression experiment were performed using groups of six-week old female Swiss mice (Janvier, Le Genest Saint Isle, France). Intramuscular of ABC/DNA formulations were injected bilaterally into both shaved tibial anterior muscles. Injected muscles were harvested 7 days after injection, frozen in liquid nitrogen and stored at −80° C. until assayed for luciferase activity.

Luciferase activity in injected muscles was analyzed as described in Pitard B, et al 0.2002. Human Gene Therapy 13:1767-75. Results are shown in FIG. 6.

### Example 4: Evaluation of Novel MCK Desmin Muscle Promoters in Nanoplasmid Vector Backbones

Extending from the surprising observation disclosed herein that 1 or 3 copies of the MCK CK7 enhance can substitute for SK-CRM4 in the Sk-CRM4-Des promoter (Table 1: Sarcar et al. Supra 2019), the incorporation of the CRE02 (SEQ ID NO:15) and CRE64 (SEQ ID NO: 16) enhancers that improved expression from the Sk-CRM4-Des promoter (Table 1: Chuah and Vanderdriessche WO2018178067) are expected to also improve expression from the MCK-Desmin promoters of the current disclosure. We further contemplate that addition of Sk-CRM4 enhancer (SEQ ID NO: 14) upstream of the MCK-Desmin promoters disclosed herein may also improve expression from the MCK-Desmin promoters of the current disclosure. We further contemplate that addition of αMHC E enhancer (SEQ ID NO: 13) upstream of the MCK-Desmin promoters disclosed herein may also improve expression from the MCK-Desmin promoters of the current disclosure, similarly to its improving expression from the MCK promoter when positioned upstream of the CK7 enhancer in the MHCK7 promoter (Table 1). These contemplated promoters would be tested as described in Examples 1 and 3 and will be of the configuration:
a) a mammalian desmin promoter element;
b) a mammalian desmin enhancer element;
c) a mammalian MCK enhancer; and
d) a mammalian enhancer element selected from the group consisting of SEQ ID NO: 13, SEQ ID NO: 14, SEQ ID NO: 15, and SEQ ID NO: 16, where the promoter and enhancer elements are operably linked.

All references, including publications, patent applications, and patents, cited herein are hereby incorporated by reference to the same extent as if each reference were individually and specifically indicated to be incorporated by reference and were set forth in its entirety herein.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

SEQUENCE LISTING

| SEQ | | | | |
|---|---|---|---|---|
| ID | | | Size | |
| NO. | Name | Gene | (bp) | Sequence |
| 1 | mMCKE | murine MCK | 143 | agccactacgggtctaggctgcccatgtaaggagg |
| | CK7 | | | caaggcctggggacacccgagatgcctggttataat |
| | Enhancer | | | taacccagacatgtggctgcccccccccccccaac |
| | | | | acctgctgcctgctaaaaataaccctgtccctggtgg |
| 2 | mMCKE | murine MCK | 434 | ccactacgggtctaggctgcccatgtaaggaggca |
| | CK7 (3x) | | | aggcctggggacacccgagatgcctggttataatta |
| | Enhancer | | | acccagacatgtggctgcccccccccccccaacac |
| | | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggt |
| 3 | mDesminE | murine | 304 | gcttcctagctgggcctttccttctcctctataaatacc |
| | (−895-−592) | desmin | | agctctggtatttcgccttggcagctgttgctgctagg |
| | Enhancer | | | gagacggctggcttgacatgcatctcctgacaaaac |
| | | | | acaaacccgtggtgtgagtgggtgtgggcggtgtg |
| | | | | agtagggggatgaatcagagagggggcgaggga |
| | | | | gacaggggcgcaggagtcaggcaaaggcgatgc |
| | | | | gggggtgcgactacacgcagttggaaacagtcgtc |
| | | | | agaagattctggaaactatcttgctggctataaacttg |
| | | | | agggaagcagaaggcca |
| 4 | mDesminP | murine | 674 | acattcctcccaagggaaactgaggctcagagttaa |
| | (−591-+83) | desmin | | aacccaggtatcagtgatatgcatgtgccccggcca |
| | promoter | | | gggtcactctctgactaaccggtacctaccctacagg |
| | | | | cctacctagagactcttttgaaaggatggtagagacc |
| | | | | tgtccgggctttgcccacagtcgttggaaacctcagc |
| | | | | attttctaggcaacttgtgcgaataaaacacttcgggg |
| | | | | gtccttcttgttcattccaataacctaaaacctctcctc |
| | | | | ggagaaaatagggggcctcaaacaaacgaaattct |
| | | | | ctagcccgctttccccaggataaggcaggcatccaa |
| | | | | atggaaaaaaaggggccggccgggggtctcctgtc |
| | | | | agctccttgccctgtgaaacccagcaggcctgcctg |
| | | | | tcttctgtcctcttggggctgtccaggggcgcaggcc |
| | | | | tcttgcgggggagctggcctccccgccccctcgcct |
| | | | | gtggccgcccttttcctggcaggacagagggatcct |
| | | | | gcagctgtcaggggaggggcgccggggggtgatg |
| | | | | tcaggagggctacaaatagtgcagacagctaaggg |
| | | | | gctccgtcacccatcttcacatccactccagccggct |
| | | | | gcccgcccgctgcctcctctgtgcgtccgcccagcc |
| | | | | agcctcgtccacgccgccacc |
| 5 | mDesminINR | murine | 670 | acattcctcccaagggaaactgaggctcagagttaa |
| | P | desmin | | aacccaggtatcagtgatatgcatgtgccccggcca |
| | (−591-+83) | | | gggtcactctctgactaaccggtacctaccctacagg |
| | promoter | | | cctacctagagactcttttgaaaggatggtagagacc |
| | | | | tgtccgggctttgcccacagtcgttggaaacctcagc |
| | | | | attttctaggcaacttgtgcgaataaaacacttcgggg |
| | | | | gtccttcttgttcattccaataacctaaaacctctcctc |
| | | | | ggagaaaatagggggcctcaaacaaacgaaattct |
| | | | | ctagcccgctttccccaggataaggcaggcatccaa |
| | | | | atggaaaaaaaggggccggccgggggtctcctgtc |
| | | | | agctccttgccctgtgaaacccagcaggcctgcctg |
| | | | | tcttctgtcctcttggggctgtccaggggcgcaggcc |
| | | | | tcttgcgggggagctggcctccccgccccctcgcct |
| | | | | gtggccgcccttttcctggcaggacagagggatcct |
| | | | | gcagctgtcaggggaggggcgccggggggtgatg |
| | | | | tcaggagggctataaaaggtgagctcgtttagtgaa |
| | | | | ccgtcagtccgcctggagacctcgagccgagcggt |
| | | | | cgtccgctgcctcctctgtgcgtccgcccagccagc |
| | | | | ctcgtccacgccgccacc |
| 6 | hDesminE | human | 281 | ggtaccccctgccccccacagctcctctcctgtgcct |
| | (−990-−620) | desmin | | tgtttcccagccatgcgttctcctctataaatacccgct |
| | Enhancer | | | ctggtatttggggttggcagctgttgctgccagggag |
| | | | | atggttgggttgacatgcggctcctgacaaaacaca |
| | | | | aacccctggtgtgtgtgggcgtgggtggtgtgagta |
| | | | | gggggatgaatcagggagggggcgggggaccca |
| | | | | gggggcaggagccacacaaagtctgtgcgggggt |
| | | | | gggagcgcacatagcaattggaaactgaa |
| 7 | hDesminP | human | 791 | agcttatcagaccctttctggaaatcagcccactgttt |
| | (−619-+86) | desmin | | ataaacttgaggccccaccctcgacagtaccgggg |
| | promoter | | | aggaagagggcctgcactagtccagagggaaact |
| | | | | gaggctcagggctagctcgcccatagacatacatg |
| | | | | gcaggcaggctttggccaggatccctccgcctgcc |
| | | | | aggcgtctccctgccctcccttcctgcctagagaccc |
| | | | | ccaccctcaagcctggctggtctttgcctgagaccca |
| | | | | aacctcttcgacttcaagagaatatttaggaacaagg |
| | | | | tggtttagggcctttcctgggaacaggccttgaccctt |
| | | | | taagaaatgacccaaagtctctccttgaccaaaaag |
| | | | | gggaccctcaaactaaagggaagcctctcttctgct |
| | | | | gtctcccctgaccccactcccccccaccccaggac |
| | | | | gaggagataaccagggctgaaagaggcccgcctg |
| | | | | ggggctgcagacatgcttgctgcctgccctggcgaa |
| | | | | ggattggcaggcttgcccgtcacaggacccccgct |
| | | | | ggctgactcaggggcgcaggcctcttgcggggga |
| | | | | gctggcctccccgcccccacggccacgggccgcc |
| | | | | ctttcctggcaggacagcgggatcttgcagctgtca |
| | | | | ggggaggggaggcgggggctgatgtcaggaggg |
| | | | | atacaaatagtgccgacggctgggggccctgtctcc |
| | | | | cctcgccgcatccactctccggccggccgcctgcc |
| | | | | cgccgcctcctccgtgcgcccgccagcctcgcccg |
| | | | | cgccgtcacc |
| 8 | hDesminINR | human | 786 | agcttatcagaccctttctggaaatcagcccactgttt |
| | P | desmin | | ataaacttgaggccccaccctcgacagtaccgggg |
| | (−619-+86) | | | aggaagagggcctgcactagtccagagggaaact |
| | promoter | | | gaggctcagggctagctcgcccatagacatacatg |
| | | | | gcaggcaggctttggccaggatccctccgcctgcc |
| | | | | aggcgtctccctgccctcccttcctgcctagagaccc |
| | | | | ccaccctcaagcctggctggtctttgcctgagaccca |
| | | | | aacctcttcgacttcaagagaatatttaggaacaagg |
| | | | | tggtttagggcctttcctgggaacaggccttgaccctt |
| | | | | taagaaatgacccaaagtctctccttgaccaaaaag |
| | | | | gggaccctcaaactaaagggaagcctctcttctgct |
| | | | | gtctcccctgaccccactcccccccaccccaggac |
| | | | | gaggagataaccagggctgaaagaggcccgcctg |
| | | | | ggggctgcagacatgcttgctgcctgccctggcgaa |
| | | | | ggattggcaggcttgcccgtcacaggacccccgct |
| | | | | ggctgactcaggggcgcaggcctcttgcggggga |
| | | | | gctggcctccccgcccccacggccacgggccgcc |
| | | | | ctttcctggcaggacagcgggatcttgcagctgtca |
| | | | | ggggaggggaggcgggggctgatgtcaggaggg |
| | | | | atataaaaggtgagctcgtttagtgaaccgtcagtcc |
| | | | | gcctggagacctcgagccgagcggtcgtccgccg |
| | | | | cctcctccgtgcgcccgccagcctcgcccgcgccg |
| | | | | tcacc |
| 9 | hDesminSP | human | 319 | agctgacatgcttgctgcctgccctggcgaaggatt |
| | (−619-+86) | desmin | | ggcaggcttgcccgtcacaggacccccgctggctg |
| | promoter | | | actcaggggcgcaggcctcttgcgggggagctgg |
| | | | | cctccccgcccccacggccacgggccgccctttcct |
| | | | | ggcaggacagcgggatcttgcagctgtcagggga |
| | | | | ggggaggcgggggctgatgtcaggagggatacaa |
| | | | | atagtgccgacggctgggggccctgtctcccctcgc |
| | | | | cgcatccactctccggccggccgcctgcccgccgc |
| | | | | ctcctccgtgcgcccgccagcctcgcccgcgccgt |
| | | | | cacc |
| 10 | hDesminINR | human | 314 | agctgacatgcttgctgcctgccctggcgaaggatt |
| | SP | desmin | | ggcaggcttgcccgtcacaggacccccgctggctg |
| | (−619-+86) | | | actcaggggcgcaggcctcttgcgggggagctgg |
| | promoter | | | cctccccgcccccacggccacgggccgccctttcct |
| | | | | ggcaggacagcgggatcttgcagctgtcagggga |
| | | | | ggggaggcgggggctgatgtcaggagggatataa |
| | | | | aaggtgagctcgtttagtgaaccgtcagtccgcctg |
| | | | | gagacctcgagccgagcggtcgtccgccgcctcct |
| | | | | ccgtgcgcccgccagcctcgcccgcgccgtcacc |
| 11 | mMCKE | murine MCK | 206 | agccactacgggtctaggctgcccatgtaaggagg |
| | Enhancer | | | caaggcctggggacacccgagatgcctggttataat |
| | | | | taacccagacatgtggctgcccccccccccccaac |
| | | | | acctgctgcctgagcctcacccccaccccggtgcct |
| | | | | gggtcttaggctctgtacaccatggaggagaagctc |
| | | | | gctctaaaaataaccctgtccctggtgg |
| 12 | hMCKE | human MCK | 255 | cccagcccccttccccgggaggtgggagcggcca |
| | Enhancer | | | cccagggccccgtggctgcccttgtaaggaggcga |
| | | | | ggcccgaggacacccgagacgcccggttataatta |
| | | | | accaggacacgtggcgaacccccctccaacacctg |
| | | | | cccccgaacccccccatacccagcgcctcgggtct |
| | | | | cggcctttgcggcagaggagacagcaaagcgccct |
| | | | | ctaaaaataactcctttcccggcgaccgagaccctcc |
| | | | | ctgtccccc |
| 13 | αMHC E | murine | 192 | acccttcagattaaaaataactgaggtaagggcctg |
| | Enhancer | aMHC | | ggtaggggaggtggtgtgagacgctcctgtctctcc |
| | | | | tctatctgcccatcggccctttggggaggaggaatgt |
| | | | | gcccaaggactaaaaaaaggccatggagccagag |
| | | | | gggcgagggcaacagacctttcatgggcaaacctt |
| | | | | ggggccctgctgtc |
| 14 | SK-CRM4 | human | 435 | ttctgagtcctctaaggtccctcactcccaactcagcc |
| | Enhancer | myosin light | | ccatgtcctgtcaattcccactcagtgtctgatctcctt |
| | | chain | | ctcctcacctttcccatctcccgtttgacccaagcttcc |
| | | | | tgagctctcctcccattcccctttttggagtcctcctcct |
| | | | | ctcccagaacccagtaataagtgggctcctccctgg |
| | | | | cctggacccccgtggtaaccctataaggcgaggca |
| | | | | gctgctgtctgaggcagggaggggctggtgtggga |
| | | | | ggctaagggcagctgctaagtttagggtggctccttc |
| | | | | tctcttcttagagacaacaggtggctggggcctcagt |
| | | | | gcccagaaaagaaaatgtcttagaggtatcggcatg |
| | | | | ggcctggaggaggggggacagggcagggggag |
| | | | | gcatcttcctcaggacatcgggtcctagagg |
| 15 | CRE02 | human | 452 | gacaggtgcggttcccggagcgcaggcgcacaca |
| | Enhancer | skeletal | | tgcacccaccggcgaacgcggtgaccctcgcccca |
| | | muscle actin | | ccccatcccctccggcgggcaactgggtcgggtca |
| | | (ACTA1) | | ggaggggcaaacccgctagggagacactccatata |
| | | | | cggcccggcccgcgttacctgggaccgggccaac |
| | | | | ccgctccttctttggtcaacgcaggggacccgggcg |
| | | | | ggggcccaggccgcgaaccggccgagggaggg |
| | | | | ggctctagtgcccaacacccaaatatggctcgagaa |
| | | | | gggcagcgacattcctgcggggtggcgcggaggg |
| | | | | aatgcccgcgggctatataaaacctgagcagaggg |
| | | | | acaagcggccaccgcagcggacagcgccaagtga |
| | | | | agcctcgcttcccctccgcggcgaccagggcccga |
| | | | | gccgagagtagcagttgtagctacccgcccaggta |
| | | | | gg |
| 16 | CRE64 | ATP2A1 | 408 | gtctccgaacgcaggccccgtcgcgttaagcacaa |
| | Enhancer | | | gctggcagggcctctcctctcccttctcagatttgctc |
| | | | | cttgacatttgcctgctgcctggcggtggcaacagct |
| | | | | ggggggggcgcgcgcaggaggccccgtaaccc |
| | | | | tatccccgctccggctccctcgtgaaaccggagcttc |
| | | | | cctgccttggccgagggggagggctgcgggggcc |
| | | | | agaccgcctgcgaagaccacagggtttttcctctcg |
| | | | | ggttttggctcccgtgggatggatgtggctgtgcgg |
| | | | | ggggttggcctgagcttcgcttctaagccagcagctt |
| | | | | ggtcagggaaacctgaaagcattcccagctaatccc |
| | | | | ccaagtggtgcaagtctgtgcgcgcccatcccgctg |
| | | | | agtaaggcggtgg |
| 17 | MVM | murine | 92 | gtaagggtttaagggatggttggttggtggggtatta |
| | intron | minute virus | | atgtttaattacctggagcacctgcctgaaatcactttt |
| | | | | tttcag |
| 18 | pCI intron | hybrid- | 133 | gtaagtatcaaggttacaagacaggtttaaggaggc |
| | | Human B | | caatagaaactgggcttgtcgagacagagaagattc |
| | | Globin | | ttgcgtttctgataggcacctattggtcttactgacatc |
| | | donor- | | cactttgcctttctctccacag |
| | | Murine IgG | | |
| | | acceptor | | |
| 19 | R6K gamma | E. coli | 281 | ggcttgttgtccacaaccgttaaaccttaaaagcttta |
| | origin-6 | | | aaagccttatatattcttttttttcttataaaacttaaaacc |
| | iteron | | | ttagaggctatttaagttgctgatttatattaattttattgt |
| | | | | tcaaacatgagagcttagtacgtgaaacatgagagc |
| | | | | ttagtacgttagccatgagagcttagtacgttagccat |
| | | | | gagggtttagttcgttaaacatgagagcttagtacgtt |
| | | | | aaacatgagagcttagtacgtactatcaacaggttga |
| | | | | actgctgatc |
| 20 | 1 CpG R6K | E. coli | 281 | ggcttgttgtccacaaccattaaaccttaaaagcttta |
| | gamma | | | aaagccttatatattcttttttttcttataaaacttaaaacc |
| | origin-6 | | | ttagaggctatttaagttgctgatttatattaattttattgt |
| | iteron | | | tcaaacatgagagcttagtacgtgaaacatgagagc |
| | | | | ttagtacattagccatgagagcttagtacattagccat |
| | | | | gagggtttagttcattaaacatgagagcttagtacatt |
| | | | | aaacatgagagcttagtacatactatcaacaggttga |
| | | | | actgctgatc |
| 21 | CpG free | E. coli | 260 | Aaaccttaaaacctttaaaagccttatatattctttttttt |
| | R6K gamma | | | cttataaaacttaaaaccttagaggctatttaagttgct |
| | origin | | | gatttatattaattttattgttcaaacatgagagcttagta |
| | | | | catgaaacatgagagcttagtacattagccatgaga |
| | | | | gcttagtacattagccatgagggtttagttcattaaac |
| | | | | atgagagcttagtacattaaacatgagagcttagtac |
| | | | | atactatcaacaggttgaactgctgatc |
| 22 | R6K gamma | E. coli | 303 | ggcttgttgtccacaaccgttaaaccttaaaagcttta |
| | origin -7 | | | aaagccttatatattcttttttttcttataaaacttaaaacc |
| | iteron | | | ttagaggctatttaagttgctgatttatattaattttattgt |
| | | | | tcaaacatgagagcttagtacgtgaaacatgagagc |
| | | | | ttagtacgttagccatgagagcttagtacgttagccat |
| | | | | gagggtttagttcgttaaacatgagagcttagtacgtt |
| | | | | aaacatgagagcttagtacgttaaacatgagagctta |
| | | | | gtacgtactatcaacaggttgaactgctgatc |
| 23 | 1 CpG R6K | E. coli | 303 | ggcttgttgtccacaaccattaaaccttaaaagcttta |
| | gamma | | | aaagccttatatattcttttttttcttataaaacttaaaacc |
| | origin-7 | | | ttagaggctatttaagttgctgatttatattaattttattgt |
| | iteron | | | tcaaacatgagagcttagtacgtgaaacatgagagc |
| | | | | ttagtacattagccatgagagcttagtacattagccat |
| | | | | gagggtttagttcattaaacatgagagcttagtacatt |
| | | | | aaacatgagagcttagtacattaaacatgagagctta |
| | | | | gtacatactatcaacaggttgaactgctgatc |
| 24 | RNA-OUT | E. coli | 239 | Gtagaattggtaaagagagtcgtgtaaaatatcgag |
| | selectable | | | ttcgcacatcttgttgtctgattattgatttttggcgaaa |
| | marker | | | ccatttgatcatatgacaagatgtgtatctaccttaactt |
| | | | | aatgattttgataaaaatcatta |
| 25 | RNA-OUT | E. coli | 69 | tcgcacatcttgttgtctgattattgatttttggcgaaac |
| | antisense | | | catttgatcatatgacaagatgtgtatct |
| | repressor | | | |
| | RNA | | | |
| 26 | 2 CpG | E. coli | 139 | gtagaattggtaaagagagttgtgtaaaatattgagtt |
| | RNA-OUT | | | cgcacatcttgttgtctgattattgatttttggcgaaacc |
| | selectable | | | atttgatcatatgacaagatgtgtatctaccttaacttaa |
| | marker | | | tgattttgataaaaatcatta |
| 27 | R6K gamma | E. coli | 431 | ggcttgttgtccacaaccgttaaaccttaaaagcttta |
| | origin-6 | | | aaagccttatatattcttttttttcttataaaacttaaaacc |
| | iteron- | | | ttagaggctatttaagttgctgatttatattaattttattgt |
| | RNA-OUT | | | tcaaacatgagagcttagtacgtgaaacatgagagc |
| | bacterial | | | ttagtacgttagccatgagagcttagtacgttagccat |
| | replication- | | | gagggtttagttcgttaaacatgagagcttagtacgtt |
| | selection | | | aaacatgagagcttagtacgtactatcaacaggttga |
| | region | | | actgctgatccacgttgtggtagaattggtaaagaga |
| | | | | gtcgtgtaaaatatcgagttcgcacatcttgttgtctga |
| | | | | ttattgatttttggcgaaaccatttgatcatatgacaag |
| | | | | atgtgtatctaccttaacttaatgattttgataaaaatca |
| | | | | ttagg |
| 28 | 1 CpG R6K | E. coli | 428 | ggcttgttgtccacaaccattaaaccttaaaagcttta |
| | gamma | | | aaagccttatatattcttttttttcttataaaacttaaaacc |
| | origin-6 | | | ttagaggctatttaagttgctgatttatattaattttattgt |
| | iteron-2 | | | tcaaacatgagagcttagtacgtgaaacatgagagc |
| | CpG RNA- | | | ttagtacattagccatgagagcttagtacattagccat |
| | OUT | | | gagggtttagttcattaaacatgagagcttagtacatt |
| | bacterial | | | aaacatgagagcttagtacatactatcaacaggttga |
| | replication- | | | actgctgatctgtacagtagaattggtaaagagagtt |
| | selection | | | gtgtaaaatattgagttcgcacatcttgttgtctgattat |
| | | | | tgatttttggcgaaaccatttgatcatatgacaagatgt |
| | | | | gtatctaccttaacttaatgattttgataaaaatcattag |
| | | | | g |
| 29 | R6K gamma | E. coli | 453 | ggcttgttgtccacaaccgttaaaccttaaaagcttta |
| | origin- | | | aaagccttatatattcttttttttcttataaaacttaaaacc |
| | 7iteron- | | | ttagaggctatttaagttgctgatttatattaattttattgt |
| | RNA-OUT | | | tcaaacatgagagcttagtacgtgaaacatgagagc |
| | bacterial | | | ttagtacgttagccatgagagcttagtacgttagccat |
| | replication- | | | gagggtttagttcgttaaacatgagagcttagtacgtt |
| | selection | | | aaacatgagagcttagtacgttaaacatgagagctta |
| | region | | | gtacgtactatcaacaggttgaactgctgatccacgtt |
| | | | | gtggtagaattggtaaagagagtcgtgtaaaatatcg |
| | | | | agttcgcacatcttgttgtctgattattgatttttggcga |
| | | | | aaccatttgatcatatgacaagatgtgtatctaccttaa |
| | | | | cttaatgattttgataaaaatcattagg |
| 30 | 1 CpG R6K | E. coli | 450 | ggcttgttgtccacaaccattaaaccttaaaagcttta |
| | gamma | | | aaagccttatatattcttttttttcttataaaacttaaaacc |
| | origin-7 | | | ttagaggctatttaagttgctgatttatattaattttattgt |
| | iteron-2 | | | tcaaacatgagagcttagtacgtgaaacatgagagc |
| | CpG RNA- | | | ttagtacattagccatgagagcttagtacattagccat |
| | OUT | | | gagggtttagttcattaaacatgagagcttagtacatt |
| | bacterial | | | aaacatgagagcttagtacattaaacatgagagctta |
| | replication- | | | gtacatactatcaacaggttgaactgctgatctgtaca |
| | selection | | | gtagaattggtaaagagagttgtgtaaaatattgagtt |
| | | | | cgcacatcttgttgtctgattattgatttttggcgaaacc |
| | | | | atttgatcatatgacaagatgtgtatctaccttaacttaa |
| | | | | tgattttgataaaaatcattagg |
| 31 | MCK- | synthetic | 1671 | ccactacgggtctaggctgcccatgtaaggaggca |
| | hDesmin- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agcttatcagaccctttctggaaatcagcccactgttt |
| | | | | ataaacttgaggccccaccctcgacagtaccgggg |
| | | | | aggaagagggcctgcactagtccagagggaaact |
| | | | | gaggctcagggctagctcgcccatagacatacatg |
| | | | | gcaggcaggctttggccaggatccctccgcctgcc |
| | | | | aggcgtctccctgccctcccttcctgcctagagaccc |
| | | | | ccaccctcaagcctggctggtctttgcctgagaccca |
| | | | | aacctcttcgacttcaagagaatatttaggaacaagg |
| | | | | tggtttagggcctttcctgggaacaggccttgaccctt |
| | | | | taagaaatgacccaaagtctctccttgaccaaaaag |
| | | | | gggaccctcaaactaaagggaagcctctcttctgct |
| | | | | gtctcccctgaccccactcccccccaccccaggac |
| | | | | gaggagataaccagggctgaaagaggcccgcctg |
| | | | | ggggctgcagacatgcttgctgcctgccctggcgaa |
| | | | | ggattggcaggcttgcccgtcacaggacccccgct |
| | | | | ggctgactcaggggcgcaggcctcttgcggggga |
| | | | | gctggcctccccgcccccacggccacgggccgcc |
| | | | | ctttcctggcaggacagcgggatcttgcagctgtca |
| | | | | ggggaggggaggcgggggctgatgtcaggaggg |
| | | | | atacaaatagtgccgacggctgggggccctgtctcc |
| | | | | cctcgccgcatccactctccggccggccgcctgcc |
| | | | | cgccgcctcctccgtgcgcccgccagcctcgcccg |
| | | | | cgccgtcacctctagaaagaggtaagggtttaaggg |
| | | | | atggttggttggtggggtattaatgtttaattacctgga |
| | | | | gcacctgcctgaaatcactttttttcagg |
| 32 | MCK- | synthetic | 1666 | ccactacgggtctaggctgcccatgtaaggaggca |
| | hDesminINR- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agcttatcagaccctttctggaaatcagcccactgttt |
| | | | | ataaacttgaggccccaccctcgacagtaccgggg |
| | | | | aggaagagggcctgcactagtccagagggaaact |
| | | | | gaggctcagggctagctcgcccatagacatacatg |
| | | | | gcaggcaggctttggccaggatccctccgcctgcc |
| | | | | aggcgtctccctgccctcccttcctgcctagagaccc |
| | | | | ccaccctcaagcctggctggtctttgcctgagaccca |
| | | | | aacctcttcgacttcaagagaatatttaggaacaagg |
| | | | | tggtttagggcctttcctgggaacaggccttgaccctt |
| | | | | taagaaatgacccaaagtctctccttgaccaaaaag |
| | | | | gggaccctcaaactaaagggaagcctctcttctgct |
| | | | | gtctcccctgaccccactcccccccaccccaggac |
| | | | | gaggagataaccagggctgaaagaggcccgcctg |
| | | | | ggggctgcagacatgcttgctgcctgccctggcgaa |
| | | | | ggattggcaggcttgcccgtcacaggacccccgct |
| | | | | ggctgactcaggggcgcaggcctcttgcggggga |
| | | | | gctggcctccccgcccccacggccacgggccgcc |
| | | | | ctttcctggcaggacagcgggatcttgcagctgtca |
| | | | | ggggaggggaggcgggggctgatgtcaggaggg |
| | | | | atataaaaggtgagctcgtttagtgaaccgtcagtcc |
| | | | | gcctggagacctcgagccgagcggtcgtccgccg |
| | | | | cctcctccgtgcgcccgccagcctcgcccgcgccg |
| | | | | tcacctctagaaagaggtaagggtttaagggatggtt |
| | | | | ggttggtggggtattaatgtttaattacctggagcacc |
| | | | | tgcctgaaatcactttttttcagg |
| 33 | MCK- | synthetic | 1199 | ccactacgggtctaggctgcccatgtaaggaggca |
| | hDesminS- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agctgacatgcttgctgcctgccctggcgaaggatt |
| | | | | ggcaggcttgcccgtcacaggacccccgctggctg |
| | | | | actcaggggcgcaggcctcttgcgggggagctgg |
| | | | | cctccccgcccccacggccacgggccgccctttcct |
| | | | | ggcaggacagcgggatcttgcagctgtcagggga |
| | | | | ggggaggcgggggctgatgtcaggagggatacaa |
| | | | | atagtgccgacggctgggggccctgtctcccctcgc |
| | | | | cgcatccactctccggccggccgcctgcccgccgc |
| | | | | ctcctccgtgcgcccgccagcctcgcccgcgccgt |
| | | | | cacctctagaaagaggtaagggtttaagggatggtt |
| | | | | ggttggtggggtattaatgtttaattacctggagcacc |
| | | | | tgcctgaaatcactttttttcagg |
| 34 | MCK- | synthetic | 1194 | ccactacgggtctaggctgcccatgtaaggaggca |
| | hDesminSINR- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agctgacatgcttgctgcctgccctggcgaaggatt |
| | | | | ggcaggcttgcccgtcacaggacccccgctggctg |
| | | | | actcaggggcgcaggcctcttgcgggggagctgg |
| | | | | cctccccgcccccacggccacgggccgccctttcct |
| | | | | ggcaggacagcgggatcttgcagctgtcagggga |
| | | | | ggggaggcgggggctgatgtcaggagggatataa |
| | | | | aaggtgagctcgtttagtgaaccgtcagtccgcctg |
| | | | | gagacctcgagccgagcggtcgtccgccgcctcct |
| | | | | ccgtgcgcccgccagcctcgcccgcgccgtcacct |
| | | | | ctagaaagaggtaagggtttaagggatggttggttg |
| | | | | gtggggtattaatgtttaattacctggagcacctgcct |
| | | | | gaaatcactttttttcagg |
| 35 | MCK- | synthetic | 1717 | ccactacgggtctaggctgcccatgtaaggaggca |
| | hDesminINR- | | | aggcctggggacacccgagatgcctggttataatta |
| | pCI | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agcttatcagaccctttctggaaatcagcccactgttt |
| | | | | ataaacttgaggccccaccctcgacagtaccgggg |
| | | | | aggaagagggcctgcactagtccagagggaaact |
| | | | | gaggctcagggctagctcgcccatagacatacatg |
| | | | | gcaggcaggctttggccaggatccctccgcctgcc |
| | | | | aggcgtctccctgccctcccttcctgcctagagaccc |
| | | | | ccaccctcaagcctggctggtctttgcctgagaccca |
| | | | | aacctcttcgacttcaagagaatatttaggaacaagg |
| | | | | tggtttagggcctttcctgggaacaggccttgaccctt |
| | | | | taagaaatgacccaaagtctctccttgaccaaaaag |
| | | | | gggaccctcaaactaaagggaagcctctcttctgct |
| | | | | gtctcccctgaccccactcccccccaccccaggac |
| | | | | gaggagataaccagggctgaaagaggcccgcctg |
| | | | | ggggctgcagacatgcttgctgcctgccctggcgaa |
| | | | | ggattggcaggcttgcccgtcacaggacccccgct |
| | | | | ggctgactcaggggcgcaggcctcttgcggggga |
| | | | | gctggcctccccgcccccacggccacgggccgcc |
| | | | | ctttcctggcaggacagcgggatcttgcagctgtca |
| | | | | ggggaggggaggcgggggctgatgtcaggaggg |
| | | | | atataaaaggtgagctcgtttagtgaaccgtcagtcc |
| | | | | gcctggagacctcgagccgagcggtcgtccgccg |
| | | | | cctcctccgtgcgcccgccagcctcgcccgcgccg |
| | | | | tcacctctagacacaggtaagtatcaaggttacaaga |
| | | | | caggtttaaggaggccaatagaaactgggcttgtcg |
| | | | | agacagagaagattcttgcgtttctgataggcacctat |
| | | | | tggtcttactgacatccactttgcctttctctccacagg |
| 36 | MCK- | synthetic | 1244 | ccactacgggtctaggctgcccatgtaaggaggca |
| | hDesminSINR- | | | aggcctggggacacccgagatgcctggttataatta |
| | pCI | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggcaagggcagggggagg |
| | | | | caccttcctcaggacatcgggtcctagagggacctt |
| | | | | gctcaggtaccccctgccccccacagctcctctcct |
| | | | | gtgccttgtttcccagccatgcgttctcctctataaata |
| | | | | cccgctctggtatttggggttggcagctgttgctgcc |
| | | | | agggagatggttgggttgacatgcggctcctgacaa |
| | | | | aacacaaacccctggtgtgtgtgggcgtgggtggtg |
| | | | | tgagtagggggatgaatcagggagggggcgggg |
| | | | | gacccagggggcaggagccacacaaagtctgtgc |
| | | | | gggggtgggagcgcacatagcaattggaaactgaa |
| | | | | agctacatgcttgctgcctgccctggcgaaggattg |
| | | | | gcaggcttgcccgtcacaggacccccgctggctga |
| | | | | ctcaggggcgcaggcctcttgcgggggagctggc |
| | | | | ctccccgcccccacggccacgggccgccctttcct |
| | | | | ggcaggacagcgggatcttgcagctgtcagggga |
| | | | | ggggaggcgggggctgatgtcaggagggatataa |
| | | | | aaggtgagctcgtttagtgaaccgtcagtccgcctg |
| | | | | gagacctcgagccgagcggtcgtccgccgcctcct |
| | | | | ccgtgcgcccgccagcctcgcccgcgccgtcacct |
| | | | | ctagacacaggtaagtatcaaggttacaagacaggt |
| | | | | ttaaggaggccaatagaaactgggcttgtcgagaca |
| | | | | gagaagattcttgcgtttctgataggcacctattggtct |
| | | | | tactgacatccactttgcctttctctccacagg |
| 37 | Murine | synthetic | 211 | ccactacgggtctaggctgcccatgtaaggaggca |
| | mMCKE- | | | aggcctggggacacccgagatgcctggttataatta |
| | 2RS5 | | | accccaacacctgctgcccccccccccccaacacc |
| | enhancer | | | tgctgcctgagcctgagcggttaccccaccccggtg |
| | | | | cctgggtcttaggctctgtacaccatggaggagaag |
| | | | | ctcgctctaaaaataaccctgtccctggtggat |
| 38 | (mMCKE- | synthetic | 633 | ccactacgggtctaggctgcccatgtaaggaggca |
| | 2RS5) 3x | | | aggcctggggacacccgagatgcctggttataatta |
| | enhancer | | | accccaacacctgctgcccccccccccccaacacc |
| | | | | tgctgcctgagcctgagcggttaccccaccccggtg |
| | | | | cctgggtcttaggctctgtacaccatggaggagaag |
| | | | | ctcgctctaaaaataaccctgtccctggtggatccact |
| | | | | acgggtctaggctgcccatgtaaggaggcaaggcc |
| | | | | tggggacacccgagatgcctggttataattaacccc |
| | | | | aacacctgctgcccccccccccccaacacctgctgc |
| | | | | ctgagcctgagcggttaccccaccccggtgcctgg |
| | | | | gtcttaggctctgtacaccatggaggagaagctcgct |
| | | | | ctaaaaataaccctgtccctggtggatccactacggg |
| | | | | tctaggctgcccatgtaaggaggcaaggcctgggg |
| | | | | acacccgagatgcctggttataattaaccccaacacc |
| | | | | tgctgcccccccccccccaacacctgctgcctgagc |
| | | | | ctgagcggttaccccaccccggtgcctgggtcttag |
| | | | | gctctgtacaccatggaggagaagctcgctctaaaa |
| | | | | ataaccctgtccctggtggat |
| 39 | mMCK | murine MCK | 133 | cctccctggggacagcccctcctggctagtcacacc |
| | promoter | | | ctgtaggctcctctatataacccaggggcacagggg |
| | (−80-+7) | | | ctgccctcattctaccaccacctccacagcacagac |
| | | | | agacactcaggagccagccagccag |
| 40 | mMCK | murine MCK | 407 | Aatcaaggctgtgggggactgagggcaggctgta |
| | promoter | | | acaggcttgggggccagggcttatacgtgcctggg |
| | (−357-+7) | | | actcccaaagtattactgttccatgttcccggcgaag |
| | | | | ggccagctgtcccccgccagctagactcagcactta |
| | | | | gtttaggaaccagtgagcaagtcagcccttggggca |
| | | | | gcccatacaaggccatggggctgggcaagctgcac |
| | | | | gcctgggtccggggtgggcacggtgcccgggcaa |
| | | | | cgagctgaaagctcatctgctctcaggggcccctcc |
| | | | | ctggggacagcccctcctggctagtcacaccctgta |
| | | | | ggctcctctatataacccaggggcacaggggctgc |
| | | | | ccccgggtcaccaccacctccacagcacagacaga |
| | | | | cactcaggagccagccag |
| 41 | MCK SIE | murine MCK | 96 | ccagcccacctgtcccaatgctgacttagtgcaagg |
| | | | | cgagccagcaaggagggaggacaggtggcagtg |
| | | | | gggggtgaggagcatctaaaaatagcc |
| 42 | MVM- | synthetic | 179 | gtaagggtttaagggatggttggttggtggggtcca |
| | MCK-SIE | | | gcccacctgtcccaatgctgacttagtgcaaggcga |
| | intron | | | gccagcaaggagggaggacaggtggcagtgggg |
| | | | | ggtgaggagcatctaaaaatagcctattaatgtttaat |
| | | | | tacctggagcacctgcctgaaatcactttttttcag |
| 43 | mMCK | synthetic | 133 | cctccctggggacagcccctcctggctagtcacacc |
| | promoter | | | ctgtaggctcctctatataacccaggggcacagggg |
| | (−80-+7) INR | | | ctgccctcattctaccaccacctccacagcacagac |
| | | | | agacactcaggagccagccagccag |
| 44 | SV40 | SV40 | 212 | Ctgtggaatgtgtgtcagttagggtgtggaaagtcc |
| | Enhancer | | | ccaggctccccagcaggcagaagtatgcaaagcat |
| | | | | gcatctcaattagtcagcaaccaggtgtggaaagtc |
| | | | | cccaggctccccagcaggcagaagtatgcaaagca |
| | | | | tgcatctcaattagtcagcaaccatagtcccgcccct |
| | | | | aactccgcccatcccgcccctaactccgcccag |
| 45 | CMV | CMV | 637 | ttacggggtcattagttcatagcccatatatggagttc |
| | Enhancer- | | | cgcgttacataacttacggtaaatggcccgcctggct |
| | promoter- | | | gaccgcccaacgacccccgcccattgacgtcaata |
| | exon 1 | | | atgacgtatgttcccatagtaacgccaatagggacttt |
| | | | | ccattgacgtcaatgggtggagtatttacggtaaact |
| | | | | gcccacttggcagtacatcaagtgtatcatatgccaa |
| | | | | gtacgccccctattgacgtcaatgacggtaaatggc |
| | | | | ccgcctggcattatgcccagtacatgaccttatggga |
| | | | | ctttcctacttggcagtacatctacgtattagtcatcgc |
| | | | | tattaccatggtgatgcggttttggcagtacatcaatg |
| | | | | ggcgtggatagcggtttgactcacggggatttccaa |
| | | | | gtctccaccccattgacgtcaatgggagtttgttttgg |
| | | | | caccaaaatcaacgggactttccaaaatgtcgtaaca |
| | | | | actccgccccattgacgcaaatgggcggtaggcgt |
| | | | | gtacggtgggaggtctatataagcagagctcgtttag |
| | | | | tgaaccgtcagatcgcctggagacgccatccacgct |
| | | | | gttttgacctccatagaagacaccgggaccgatcca |
| | | | | gcctccgcgg |
| 46 | MCAT | synthetic | 23 | CAGAGAGGAATGCAACACTTGC |
| | | | | A |
| 47 | MCAT (2x) | synthetic | 51 | ctagacagagaggaatgcaacacttgcacagagag |
| | | | | gaatgcaacacttgca |
| 48 | C5-12 | synthetic | 333 | ggccgtccgccctcggcaccatcctcacgacaccc |
| | enhancer | | | aaatatggcgacgggtgaggaatggtggggagttat |
| | promoter- | | | ttttagagcggtgaggaaggtgggcaggcagcagg |
| | exon 1 | | | tgttggcgctctaaaaataactcccgggagttattttta |
| | | | | gagcggaggaatggtggacacccaaatatggcga |
| | | | | cggttcctcacccgtcgccatatttgggtgtccgccct |
| | | | | cggccggggccgcattcctgggggccgggcggtg |
| | | | | ctcccgcccgcctcgataaaaggctccggggccgg |
| | | | | cggcggcccacgagctacccggaggagcgggag |
| | | | | gcgccaagctctag |
| 49 | hDesminP | human | 355 | gagataaccagggctgaaagaggcccgcctgggg |
| | (−253-+86) | desmin | | gctgcagacatgcttgctgcctgccctggcgaagga |
| | promoter | | | ttggcaggcttgcccgtcacaggacccccgctggct |
| | | | | gactcaggggcgcaggccttttgcgggggagctgg |
| | | | | cctccccgcccccacggccacgggccgccctttcct |
| | | | | ggcaggacagcgggatcttgcagctgtcagggga |
| | | | | ggggaggcgggggctgatgtcaggagggatacaa |
| | | | | atagtgccgacggctgggggccctgtctcccctcgc |
| | | | | cgcatccactctccggccggccgcctgcccgccgc |
| | | | | ctcctccgtgcgcccgccagcctcgcccgcgccgt |
| | | | | cacc |
| 50 | hDesminE | human | 359 | cacccatgcctcctcaggtaccccctgccccccaca |
| | (−1008-−558) | desmin | | gctcctctcctgtgccttgtttcccagccatgcgttctc |
| | | | | ctctataaatacccgctctggtatttggggttggcagc |
| | | | | tgttgctgccagggagatggttgggttgacatgcgg |
| | | | | ctcctgacaaaacacaaacccctggtgtgtgtgggc |
| | | | | gtgggtggtgtgagtagggggatgaatcagggagg |
| | | | | gggcaggggacccagggggcaggagccacacaa |
| | | | | agtctgtgcgggggtgggagcgcacatagcaattg |
| | | | | gaaactgaaagcttatcagaccctttctggaaatcag |
| | | | | cccactgtttataaacttgaggccccaccctcga |
| 51 | CMV | CMV | 588 | ttgacattgattattgactagttattaatagtaatcaatta |
| | enhancer- | | | cggggtcattagttcatagcccatatatggagttccg |
| | promoter- | | | cgttacataacttacggtaaatggcccgcctggctga |
| | Exon 1 | | | ccgcccaacgacccccgcccattgacgtcaataatg |
| | | | | acgtatgttcccatagtaacgccaatagggactttcc |
| | | | | attgacgtcaatgggtggactatttacggtaaactgc |
| | | | | ccacttggcagtacatcaagtgtatcatatgccaagt |
| | | | | acgccccctattgacgtcaatgacggtaaatggccc |
| | | | | gcctggcattatgcccagtacatgaccttatgggactt |
| | | | | tcctacttggcagtacatctacgtattagtcatcgctat |
| | | | | taccatggtgatgcggttttggcagtacatcaatggg |
| | | | | cgtggatagcggtttgactcacggggatttccaagtc |
| | | | | tccaccccattgacgtcaatgggagtttgttttggcac |
| | | | | caaaatcaacgggactttccaaaatgtcgtaacaact |
| | | | | ccgccccattgacgcaaatgggcggtaggcgtgta |
| | | | | cggtgggaggtctatataagcagagctct |
| 52 | hDesminE | human | 635 | acacacctactagtaacccctccagctggtgatggc |
| | (−1340-−620) | desmin | | aggtctagggtaggaccagtgactggctcctaatcg |
| | Enhancer | | | agcactctattttcagggtttgcattccaaaagggtca |
| | | | | ggtccaagagggacctggagtgccaagtggaggt |
| | | | | gtagaggcacggccagtacccatggagaatggtgg |
| | | | | atgtccttaggggttagcaagtgccgtgtgctaagga |
| | | | | gggggctttggaggttgggcaggccctctgtgggg |
| | | | | ctccatttttgtgggggtgggggctggagcattatag |
| | | | | ggggtgggaagtgattggggctgtcaccctagcctt |
| | | | | ccttatctgacgcccacccatgcctcctcaggtaccc |
| | | | | cctgccccccacagctcctctcctgtgccttgtttccc |
| | | | | agccatgcgttctcctctataaatacccgctctggtatt |
| | | | | tggggttggcagctgttgctgccagggagatggttg |
| | | | | ggttgacatgcggctcctgacaaaacacaaacccct |
| | | | | ggtgtgtgtgggcgtgggtggtgtgagtagggggat |
| | | | | gaatcagggagggggcgggggacccagggggca |
| | | | | ggagccacacaaagtctgtgcgggggtgggagcg |
| | | | | cacatagcaattggaaactgaa |
| 53 | HR B intron | synthetic | 341 | ctcgcatctctccttcacgcgcccgccgccctacctg |
| | | | | aggccgccatccacgccggttgagtcgcgttctgcc |
| | | | | gcctcccgcctgtggtgcctcctgaactgcgtccgc |
| | | | | cgtctaggtaagtttaaagctcaggtcgagaccggg |
| | | | | cctttgtccggcgctcccttggagcctacctagactc |
| | | | | agccggctctccacgctttgcctgaccctgcttgctc |
| | | | | aactctagttctctcgttaacttaatgagacagataga |
| | | | | aactggtcttgtagaaacagagtagtcgcctgcttttc |
| | | | | tgccaggtgctgacttctctcccctgggcttttttcttttt |
| | | | | ctcag |
| 54 | MCK2R | murine MCK | 142 | agccactacgggtctaggctgcccatgtaaggagg |
| | Enhancer | | | caaggcctggggacacccgagatgcctggttataat |
| | | | | taaccccaacacctgctgcccccccccccccaaca |
| | | | | cctgctgcctgctaaaaataaccctgtccctggtgg |
| 55 | MCK- | synthetic | 1572 | ccactacgggtctaggctgcccatgtaaggaggca |
| | mDesmin- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggacagggcagggggagg |
| | | | | catcttcctcaggacatcgggtcctagagggaccttg |
| | | | | cttcctagctgggcctttccttctcctctataaatacca |
| | | | | gctctggtatttcgccttggcagctgttgctgctaggg |
| | | | | agacggctggcttgacatgcatctcctgacaaaaca |
| | | | | caaacccgtggtgtgagtgggtgtgggcggtgtga |
| | | | | gtagggggatgaatcagagagggggcgagggag |
| | | | | acaggggcgcaggagtcaggcaaaggcgatgcg |
| | | | | ggggtgcgactacacgcagttggaaacagtcgtca |
| | | | | gaagattctggaaactatcttgctggctataaacttga |
| | | | | gggaagcagaaggccaacattcctcccaagggaa |
| | | | | actgaggctcagagttaaaacccaggtatcagtgat |
| | | | | atgcatgtgccccggccagggtcactctctgactaa |
| | | | | ccggtacctaccctacaggcctacctagagactctttt |
| | | | | gaaaggatggtagagacctgtccgggctttgcccac |
| | | | | agtcgttggaaacctcagcattttctaggcaacttgtg |
| | | | | cgaataaaacacttcgggggtccttcttgttcattcca |
| | | | | ataacctaaaacctctcctcggagaaaatagggggc |
| | | | | ctcaaacaaacgaaattctctagcccgctttccccag |
| | | | | gataaggcaggcatccaaatggaaaaaaaggggc |
| | | | | cggccgggggtctcctgtcagctccttgccctgtga |
| | | | | aacccagcaggcctgcctgtcttctgtcctcttgggg |
| | | | | ctgtccaggggcgcaggcctcttgcgggggagctg |
| | | | | gcctccccgccccctcgcctgtggccgcccttttcct |
| | | | | ggcaggacagagggatcctgcagctgtcagggga |
| | | | | ggggcgccggggggtgatgtcaggagggctacaa |
| | | | | atagtgcagacagctaaggggctccgtcacccatct |
| | | | | tcacatccactccagccggctgcccgcccgctgcct |
| | | | | cctctgtgcgtccgcccagccagcctcgtccacgcc |
| | | | | gccacctctagaaagaggtaagggtttaagggatgg |
| | | | | ttggttggtggggtattaatgtttaattacctggagcac |
| | | | | ctgcctgaaatcactttttttcagg |
| 56 | MCK- | synthetic | 1567 | ccactacgggtctaggctgcccatgtaaggaggca |
| | mDesminINR- | | | aggcctggggacacccgagatgcctggttataatta |
| | MVM | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctaaggaggggggacagggcagggggaggc |
| | | | | atcttcctcaggacatcgggtcctagagggaccttgc |
| | | | | ttcctagctgggcctttccttctcctctataaataccag |
| | | | | ctctggtatttcgccttggcagctgttgctgctaggga |
| | | | | gacggctggcttgacatgcatctcctgacaaaacac |
| | | | | aaacccgtggtgtgagtgggtgtgggcggtgtgagt |
| | | | | agggggatgaatcagagagggggcgagggagac |
| | | | | aggggcgcaggagtcaggcaaaggcgatgcggg |
| | | | | ggtgcgactacacgcagttggaaacagtcgtcaga |
| | | | | agattctggaaactatcttgctggctataaacttgagg |
| | | | | gaagcagaaggccaacattcctcccaagggaaact |
| | | | | gaggctcagagttaaaacccaggtatcagtgatatg |
| | | | | catgtgccccggccagggtcactctctgactaaccg |
| | | | | gtacctaccctacaggcctacctagagactcttttgaa |
| | | | | aggatggtagagacctgtccgggctttgcccacagt |
| | | | | cgttggaaacctcagcattttctaggcaacttgtgcga |
| | | | | ataaaacacttcgggggtccttcttgttcattccaataa |
| | | | | cctaaaacctctcctcggagaaaatagggggcctca |
| | | | | aacaaacgaaattctctagcccgctttccccaggata |
| | | | | aggcaggcatccaaatggaaaaaaaggggccggc |
| | | | | cgggggtctcctgtcagctccttgccctgtgaaaccc |
| | | | | agcaggcctgcctgtcttctgtcctcttggggctgtcc |
| | | | | aggggcgcaggcctcttgcgggggagctggcctc |
| | | | | cccgccccctcgcctgtggccgcccttttcctggca |
| | | | | ggacagagggatcctgcagctgtcaggggagggg |
| | | | | cgccggggggtgatgtcaggagggctataaaaggt |
| | | | | gagctcgtttagtgaaccgtcagtccgcctggagac |
| | | | | ctcgagccgagcggtcgtccgctgcctcctctgtgc |
| | | | | gtccgcccagccagcctcgtccacgccgccacctct |
| | | | | agaaagaggtaagggtttaagggatggttggttggt |
| | | | | ggggtattaatgtttaattacctggagcacctgcctga |
| | | | | aatcactttttttcagg |
| 57 | MCK- | synthetic | 1618 | ccactacgggtctaggctgcccatgtaaggaggca |
| | mDesminINR- | | | aggcctggggacacccgagatgcctggttataatta |
| | pCI | | | acccagacatgtggctgcccccccccccccaacac |
| | intron | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctaaggaggggggacagggcagggggaggc |
| | | | | atcttcctcaggacatcgggtcctagagggaccttgc |
| | | | | ttcctagctgggcctttccttctcctctataaataccag |
| | | | | ctctggtatttcgccttggcagctgttgctgctaggga |
| | | | | gacggctggcttgacatgcatctcctgacaaaacac |
| | | | | aaacccgtggtgtgagtgggtgtgggcggtgtgagt |
| | | | | agggggatgaatcagagagggggcgagggagac |
| | | | | aggggcgcaggagtcaggcaaaggcgatgcggg |
| | | | | ggtgcgactacacgcagttggaaacagtcgtcaga |
| | | | | agattctggaaactatcttgctggctataaacttgagg |
| | | | | gaagcagaaggccaacattcctcccaagggaaact |
| | | | | gaggctcagagttaaaacccaggtatcagtgatatg |
| | | | | catgtgccccggccagggtcactctctgactaaccg |
| | | | | gtacctaccctacaggcctacctagagactcttttgaa |
| | | | | aggatggtagagacctgtccgggctttgcccacagt |
| | | | | cgttggaaacctcagcattttctaggcaacttgtgcga |
| | | | | ataaaacacttcgggggtccttcttgttcattccaataa |
| | | | | cctaaaacctctcctcggagaaaatagggggcctca |
| | | | | aacaaacgaaattctctagcccgctttccccaggata |
| | | | | aggcaggcatccaaatggaaaaaaaggggccggc |
| | | | | cgggggtctcctgtcagctccttgccctgtgaaaccc |
| | | | | agcaggcctgcctgtcttctgtcctcttggggctgtcc |
| | | | | aggggcgcaggcctcttgcgggggagctggcctc |
| | | | | cccgccccctcgcctgtggccgcccttttcctggca |
| | | | | ggacagagggatcctgcagctgtcaggggagggg |
| | | | | cgccggggggtgatgtcaggagggctataaaaggt |
| | | | | gagctcgtttagtgaaccgtcagtccgcctggagac |
| | | | | ctcgagccgagcggtcgtccgctgcctcctctgtgc |
| | | | | gtccgcccagccagcctcgtccacgccgccacctct |
| | | | | agacacaggtaagtatcaaggttacaagacaggttt |
| | | | | aaggaggccaatagaaactgggcttgtcgagacag |
| | | | | agaagattcttgcgtttctgataggcacctattggtctt |
| | | | | actgacatccactttgcctttctctccacagg |
| 58 | MCK- | synthetic | 1623 | ccactacgggtctaggctgcccatgtaaggaggca |
| | mDesmin- | | | aggcctggggacacccgagatgcctggttataatta |
| | pCI intron | | | acccagacatgtggctgcccccccccccccaacac |
| | | | | ctgctgcctgctaaaaataaccctgtccctggtggtct |
| | | | | agccactacgggtctaggctgcccatgtaaggagg |
| | | | | caaggcctggggacacccgagatgcctggttataat |
| | | | | taacccagacatgtggctgcccccccccccccaac |
| | | | | acctgctgcctgctaaaaataaccctgtccctggtgg |
| | | | | tctagccactacgggtctaggctgcccatgtaagga |
| | | | | ggcaaggcctggggacacccgagatgcctggttat |
| | | | | aattaacccagacatgtggctgccccccccccccca |
| | | | | acacctgctgcctgctaaaaataaccctgtccctggt |
| | | | | ggtctagaggaggggggacagggcagggggagg |
| | | | | catcttcctcaggacatcgggtcctagagggaccttg |
| | | | | cttcctagctgggcctttccttctcctctataaatacca |
| | | | | gctctggtatttcgccttggcagctgttgctgctaggg |
| | | | | agacggctggcttgacatgcatctcctgacaaaaca |
| | | | | caaacccgtggtgtgagtgggtgtgggcggtgtga |
| | | | | gtagggggatgaatcagagagggggcgagggag |
| | | | | acaggggcgcaggagtcaggcaaaggcgatgcg |
| | | | | ggggtgcgactacacgcagttggaaacagtcgtca |
| | | | | gaagattctggaaactatcttgctggctataaacttga |
| | | | | gggaagcagaaggccaacattcctcccaagggaa |
| | | | | actgaggctcagagttaaaacccaggtatcagtgat |
| | | | | atgcatgtgccccggccagggtcactctctgactaa |
| | | | | ccggtacctaccctacaggcctacctagagactctttt |
| | | | | gaaaggatggtagagacctgtccgggctttgcccac |
| | | | | agtcgttggaaacctcagcattttctaggcaacttgtg |
| | | | | cgaataaaacacttcgggggtccttcttgttcattcca |
| | | | | ataacctaaaacctctcctcggagaaaatagggggc |
| | | | | ctcaaacaaacgaaattctctagcccgctttccccag |
| | | | | gataaggcaggcatccaaatggaaaaaaaggggc |
| | | | | cggccgggggtctcctgtcagctccttgccctgtga |
| | | | | aacccagcaggcctgcctgtcttctgtcctcttgggg |
| | | | | ctgtccaggggcgcaggcctcttgcgggggagctg |
| | | | | gcctccccgccccctcgcctgtggccgcccttttcct |
| | | | | ggcaggacagagggatcctgcagctgtcagggga |
| | | | | ggggcgccggggggtgatgtcaggagggctacaa |
| | | | | atagtgcagacagctaaggggctccgtcacccatct |
| | | | | tcacatccactccagccggctgcccgcccgctgcct |
| | | | | cctctgtgcgtccgcccagccagcctcgtccacgcc |
| | | | | gccacctctagacacaggtaagtatcaaggttacaa |
| | | | | gacaggtttaaggaggccaatagaaactgggcttgt |
| | | | | cgagacagagaagattcttgcgtttctgataggcacc |
| | | | | tattggtcttactgacatccactttgcctttctctccaca |
| | | | | gg |
| 59 | INR | synthetic | 68 | tataaaaggtgagctcgtttagtgaaccgtcagtccg |
| | | | | cctggagacctcgagccgagcggtcgtccgc |

1. A recombinant muscle-specific regulatory nucleic acid sequence, comprising:
a mammalian desmin promoter, wherein the mammalian desmin promoter comprises a nucleic acid sequence having at least 80% identity to a nucleic acid sequence selected from the group consisting of SEQ ID NO: 7, SEQ ID NO: 8, SEQ ID NO: 9, and SEQ ID NO: 10;
a mammalian desmin enhancer; and
one or more mammalian muscle creatine kinase (MCK) enhancers,
wherein the mammalian desmin promoter, mammalian desmin enhancer, and mammalian MCK enhancer are operably linked.

2. (canceled)

3. (canceled)

4. The recombinant muscle-specific regulatory nucleic acid sequence of claim 1, wherein the mammalian desmin promoter comprises a nucleic acid sequence having at least 80% identity to a nucleic acid sequence selected from the group consisting of SEQ ID NO: 8 and SEQ ID NO: 10.

5. (canceled)

6. (canceled)

7. (canceled)

8. The recombinant muscle-specific regulatory nucleic acid sequence of claim 1, wherein the mammalian desmin enhancer is a human desmin enhancer.

9. The recombinant muscle-specific regulatory nucleic acid sequence of claim 1, wherein the mammalian desmin enhancer comprises a nucleic acid sequence having at least 80% identity to the nucleic acid sequence of SEQ ID NO: 6.

10. The recombinant muscle-specific regulatory nucleic acid sequence of claim 1, wherein the mammalian desmin enhancer comprises the nucleic acid sequence of SEQ ID NO: 6.

11. (canceled)

12. (canceled)

13. (canceled)

14. The recombinant muscle-specific regulatory nucleic acid sequence of claim 1, wherein the one or more mammalian MCK enhancers are each a MCK CK7 enhancer.

15. (canceled)

16. (canceled)

17. The recombinant muscle-specific regulatory nucleic acid sequence of claim 1, wherein the one or more mammalian MCK enhancers each comprise a nucleic acid sequence of SEQ ID NO: 2.

18. (canceled)

19. The recombinant muscle-specific regulatory nucleic acid sequence of claim 1, wherein the one or more mammalian MCK enhancers each have a nucleic acid sequence with at least 80% identity to the nucleic acid sequence of SEQ ID NO: 1.

20.-49. (canceled)

50. A vector, comprising:
i) a eukaryotic region sequence comprising a muscle-specific regulatory nucleic acid sequence, wherein the muscle-specific regulatory nucleic acid sequence comprises a mammalian desmin promoter, a mammalian desmin enhancer; and one or more mammalian muscle creatine kinase (MCK) enhancers, wherein the mammalian desmin promoter, mammalian desmin enhancer, and mammalian MCK enhancer are operably linked; and
ii) a spacer region that links the 5′ and 3′ ends of the eukaryotic region sequence and that comprises a bacterial replication origin and a RNA selectable marker.

51. (canceled)

52. (canceled)

53. (canceled)

54. The vector of claim 50, wherein the bacterial replication origin is a R6K bacterial replication origin.

55. The vector of claim 54, wherein the bacterial replication origin comprises a sequence having at least 80% identity to any one of SEQ ID NOs: 19-23.

56. The vector of claim 50, wherein the RNA selectable marker comprises a sequence having at least 80% identity to SEQ ID NO: 24 or SEQ ID NO: 26.

57.-61. (canceled)

62. A recombinant muscle-specific regulatory nucleic acid sequence, comprising: a mammalian desmin promoter; a mammalian desmin enhancer, wherein the mammalian desmin enhancer comprises a nucleic acid sequence having at least 80% identity to the nucleic acid sequence of SEQ ID NO: 6; and one or more mammalian muscle creatine kinase (MCK) enhancers, wherein the mammalian desmin promoter, mammalian desmin enhancer, and mammalian MCK enhancer are operably linked.

63. The recombinant muscle-specific regulatory nucleic acid sequence of claim 62, wherein the mammalian desmin promoter comprises a nucleic acid sequence having at least 80% identity to a nucleic acid sequence selected from the group consisting of SEQ ID NO: 8 and SEQ ID NO: 10.

64. The recombinant muscle-specific regulatory nucleic acid sequence of claim 62, wherein the one or more mammalian MCK enhancers each comprise a nucleic acid sequence of SEQ ID NO: 2.

65. The recombinant muscle-specific regulatory nucleic acid sequence of claim 62, wherein the one or more mammalian MCK enhancers each have a nucleic acid sequence with at least 80% identity to a nucleic acid sequence of SEQ ID NO: 1.

66. The recombinant muscle-specific regulatory nucleic acid sequence of claim 62, wherein the mammalian desmin promoter comprises a nucleic acid sequence having at least 80% identity to a nucleic acid sequence selected from the group consisting of SEQ ID NO: 8 and SEQ ID NO: 10, and wherein the one or more mammalian MCK enhancers each comprise a nucleic acid sequence of SEQ ID NO: 2.

67. The recombinant muscle-specific regulatory nucleic acid sequence of claim 62, wherein the mammalian desmin promoter comprises a nucleic acid sequence having at least 80% identity to a nucleic acid sequence selected from the group consisting of SEQ ID NO: 8 and SEQ ID NO: 10, and wherein the one or more mammalian MCK enhancers each have a nucleic acid sequence with at least 80% identity to a nucleic acid sequence of SEQ ID NO: 1.

68. The recombinant muscle-specific regulatory nucleic acid sequence of claim 62, wherein the mammalian desmin promoter comprises a nucleic acid sequence having at least 80% identity to a nucleic acid sequence of SEQ ID NO: 8, and wherein the one or more mammalian MCK enhancers each comprise nucleic acid sequence of SEQ ID NO: 2.

69. The recombinant muscle-specific regulatory nucleic acid sequence of claim 62, wherein the mammalian desmin promoter comprises a nucleic acid sequence having at least 80% identity to a nucleic acid sequence of SEQ ID NO: 10, and wherein the one or more mammalian MCK enhancers each comprise a nucleic acid sequence of SEQ ID NO: 2.


SEQUENCE LISTING (59 sequences)

SEQ ID NO: 1 - DNA, 143 bp, Mus sp.
  source 1..143: mol_type = genomic DNA
agccactacgggtctaggctgcccatgtaaggaggcaaggcctggggacacccgagatgcctggttataattaacccagacatgtggctgcccccccccccccaacacctgctgcctgctaaaaataaccctgtccctggtgg

SEQ ID NO: 2 - DNA, 434 bp, Mus sp.
  source 1..434: mol_type = genomic DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggt

SEQ ID NO: 3 - DNA, 304 bp, Mus sp.
  source 1..304: mol_type = genomic DNA
gcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggcca

SEQ ID NO: 4 - DNA, 674 bp, Mus sp.
  source 1..674: mol_type = genomic DNA
acattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccgccacc

SEQ ID NO: 5 - DNA, 670 bp, Mus sp.
  source 1..670: mol_type = genomic DNA
acattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctataaaaggtgagctcgtttagtgaaccgtcagtccgcctggagacctcgagccgagcggtcgtccgctgcctcctctgtgcgtccgcccagccagcctcgtccacgccgccacc

SEQ ID NO: 6 - DNA, 281 bp, Homo sapiens
  source 1..281: mol_type = genomic DNA
ggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaa

SEQ ID NO: 7 - DNA, 791 bp, Homo sapiens
  source 1..791: mol_type = genomic DNA
agcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 8 - DNA, 786 bp, Homo sapiens
  source 1..786: mol_type = genomic DNA
agcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacc

SEQ ID NO: 9 - DNA, 319 bp, Homo sapiens
  source 1..319: mol_type = genomic DNA
agctgacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 10 - DNA, 314 bp, Homo sapiens
  source 1..314: mol_type = genomic DNA
agctgacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacc

SEQ ID NO: 11 - DNA, 206 bp, Mus sp.
  source 1..206: mol_type = genomic DNA
agccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtgg

SEQ ID NO: 12 - DNA, 255 bp, Homo sapiens
  source 1..255: mol_type = genomic DNA
cccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtccccc

SEQ ID NO: 13 - DNA, 192 bp, Mus sp.
  source 1..192: mol_type = genomic DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtc

SEQ ID NO: 14 - DNA, 435 bp, Homo sapiens
  source 1..435: mol_type = genomic DNA
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagg

SEQ ID NO: 15 - DNA, 452 bp, Homo sapiens
  source 1..452: mol_type = genomic DNA
gacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtagg

SEQ ID NO: 16 - DNA, 408 bp, unidentified
  misc_feature 1..408: note = Description of Unknown: ATP2A1 sequence
  source 1..408: mol_type = unassigned DNA
gtctccgaacgcaggccccgtcgcgttaagcacaagctggcagggcctctcctctcccttctcagatttgctccttgacatttgcctgctgcctggcggtggcaacagctggggcggggcgcgcgcaggaggccccgtaaccctatccccgctccggctccctcgtgaaaccggagcttccctgccttggccgagggggagggctgcgggggccagaccgcctgcgaagaccacagggtttttcctctcgggttttggctcccgtgggatggatgtggctgtgcggggggttggcctgagcttcgcttctaagccagcagcttggtcagggaaacctgaaagcattcccagctaatcccccaagtggtgcaagtctgtgcgcgcccatcccgctgagtaaggcggtgg

SEQ ID NO: 17 - DNA, 82 bp, unidentified
  misc_feature 1..82: note = Description of Unknown: murine minute virus sequence
  source 1..82: mol_type = unassigned DNA
gtaagggtttaagggatggttggttggtggggtattaatgtttaattacctggagcacctgcctgaaatcactttttttcag

SEQ ID NO: 18 - DNA, 133 bp, synthetic construct
  misc_feature 1..133: note = Description of Artificial Sequence: Synthetic polynucleotide
  source 1..133: mol_type = other DNA
gtaagtatcaaggttacaagacaggtttaaggaggccaatagaaactgggcttgtcgagacagagaagattcttgcgtttctgataggcacctattggtcttactgacatccactttgcctttctctccacag

SEQ ID NO: 19 - DNA, 281 bp, Escherichia coli
  source 1..281: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 20 - DNA, 281 bp, Escherichia coli
  source 1..281: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 21 - DNA, 260 bp, Escherichia coli
  source 1..260: mol_type = other DNA
aaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 22 - DNA, 303 bp, Escherichia coli
  source 1..303: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgttaaacatgagagcttagtacgtactatcaacaggttgaactgctgatc

SEQ ID NO: 23 - DNA, 303 bp, Escherichia coli
  source 1..303: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatc

SEQ ID NO: 24 - DNA, 139 bp, Escherichia coli
  source 1..139: mol_type = other DNA
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 25 - DNA, 69 bp, Escherichia coli
  source 1..69: mol_type = other DNA
tcgcacatcttgttgtctgattattgatttttggcgaaaccatttgatcatatgacaagatgtgtatct

SEQ ID NO: 26 - DNA, 139 bp, Escherichia coli
  source 1..139: mol_type = other DNA
gtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 27 - DNA, 431 bp, Escherichia coli
  source 1..431: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattagg

SEQ ID NO: 28 - DNA, 428 bp, Escherichia coli
  source 1..428: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattagg

SEQ ID NO: 29 - DNA, 453 bp, Escherichia coli
  source 1..453: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattagg

SEQ ID NO: 30 - DNA, 450 bp, Escherichia coli
  source 1..450: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattagg

SEQ ID NO: 31 - DNA, 1671 bp, synthetic construct
  misc_feature 1..1671: note = Description of Artificial Sequence: Synthetic polynucleotide
  source 1..1671: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcagg

SEQ ID NO: 32 - DNA, 1666 bp, synthetic construct
  misc_feature 1..1666: note = Description of Artificial Sequence: Synthetic polynucleotide
  source 1..1666: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagaaagaggtaagggtttaagggatggttggttggtggggtattaatgtttaattacctggagcacctgcctgaaatcactttttttcagg

SEQ ID NO: 33 - DNA, 1199 bp, synthetic construct
  misc_feature 1..1199: note = Description of Artificial Sequence: Synthetic polynucleotide
  source 1..1199: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctgacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcagg

SEQ ID NO: 34 - DNA, 1194 bp, synthetic construct
  misc_feature 1..1194: note = Description of Artificial Sequence: Synthetic polynucleotide
  source 1..1194: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctgacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagaaagaggtaagggtttaagggatggttggttggtggggtattaatgtttaattacctggagcacctgcctgaaatcactttttttcagg

SEQ ID NO: 35 - DNA, 1717 bp, synthetic construct
  misc_feature 1..1717: note = Description of Artificial Sequence: Synthetic polynucleotide
  source 1..1717: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacagg

SEQ ID NO: 36 - DNA, 1244 bp, synthetic construct
  misc_feature 1..1244: note = Description of Artificial Sequence: Synthetic polynucleotide
  source 1..1244: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacagg

SEQ ID NO: 37 - DNA, 211 bp, synthetic construct
  misc_feature 1..211: note = Description of Artificial Sequence: Synthetic polynucleotide
  source 1..211: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggat

SEQ ID NO: 38 - DNA, 633 bp, synthetic construct
  misc_feature 1..633: note = Description of Artificial Sequence: Synthetic polynucleotide
  source 1..633: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggat

SEQ ID NO: 39 - DNA, 133 bp, Mus sp.
  source 1..133: mol_type = genomic DNA
cctccctggggacagcccctcctggctagtcacaccctgtaggctcctctatataacccaggggcacaggggctgccctcattctaccaccacctccacagcacagacagacactcaggagccagccagccag

SEQ ID NO: 40 - DNA, 407 bp, Mus sp.
  source 1..407: mol_type = genomic DNA
aatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccag

SEQ ID NO: 41 - DNA, 96 bp, Mus sp.
  source 1..96: mol_type = genomic DNA
ccagcccacctgtcccaatgctgacttagtgcaaggcgagccagcaaggagggaggacaggtggcagtggggggtgaggagcatctaaaaatagcc

SEQ ID NO: 42 - DNA, 179 bp, synthetic construct
  misc_feature 1..179: note = Description of Artificial Sequence: Synthetic polynucleotide
  source 1..179: mol_type = other DNA
gtaagggtttaagggatggttggttggtggggtccagcccacctgtcccaatgctgacttagtgcaaggcgagccagcaaggagggaggacaggtggcagtggggggtgaggagcatctaaaaatagcctattaatgtttaattacctggagcacctgcctgaaatcactttttttcag

SEQ ID NO: 43 - DNA, 133 bp, synthetic construct; identical to SEQ ID NO: 39
  misc_feature 1..133: note = Description of Artificial Sequence: Synthetic polynucleotide
  source 1..133: mol_type = other DNA

SEQ ID NO: 44 - DNA, 212 bp, Simian virus 40
  source 1..212: mol_type = genomic DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccag

SEQ ID NO: 45 - DNA, 637 bp, Cytomegalovirus sp.
  source 1..637: mol_type = genomic DNA
ttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 46 - DNA, 23 bp, synthetic construct
  misc_feature 1..23: note = Description of Artificial Sequence: Synthetic oligonucleotide
  source 1..23: mol_type = other DNA
cagagaggaatgcaacacttgca

SEQ ID NO: 47 - DNA, 51 bp, synthetic construct
  misc_feature 1..51: note = Description of Artificial Sequence: Synthetic oligonucleotide
  source 1..51: mol_type = other DNA
ctagacagagaggaatgcaacacttgcacagagaggaatgcaacacttgca

SEQ ID NO: 48 - DNA, 333 bp, synthetic construct
  misc_feature 1..333: note = Description of Artificial Sequence: Synthetic polynucleotide
  source 1..333: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctag

SEQ ID NO: 49 - DNA, 355 bp, Homo sapiens
  source 1..355: mol_type = genomic DNA
gagataaccagggctgaaagaggcccgcctgggggctgcagacatgcttgctgcctgccctggcgaaggattggcaggcttgcccgtcacaggacccccgctggctgactcaggggcgcaggccttttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 50 - DNA, 359 bp, Homo sapiens
  source 1..359: mol_type = genomic DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcaggggacccagggggcaggagccacacaaagtctgtgcgggggtgggagcgcacatagcaattggaaactgaaagcttatcagaccctttctggaaatcagcccactgtttataaacttgaggccccaccctcga

SEQ ID NO: 51 - DNA, 588 bp, Cytomegalovirus sp.
  source 1..588: mol_type = genomic DNA
ttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctct

SEQ ID NO: 52 - DNA, 635 bp, Homo sapiens
  source 1..635: mol_type = genomic DNA
acacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaa

SEQ ID NO: 53 - DNA, 341 bp, synthetic construct
  misc_feature 1..341: note = Description of Artificial Sequence: Synthetic polynucleotide
  source 1..341: mol_type = other DNA
ctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcag

SEQ ID NO: 54 - DNA, 142 bp, Mus sp.
  source 1..142: mol_type = genomic DNA
agccactacgggtctaggctgcccatgtaaggaggcaaggcctggggacacccgagatgcctggttataattaaccccaacacctgctgcccccccccccccaacacctgctgcctgctaaaaataaccctgtccctggtgg

SEQ ID NO: 55 - DNA, 1572 bp, synthetic construct
  misc_feature 1..1572: note = Description of Artificial Sequence: Synthetic polynucleotide
  source 1..1572: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggacagggcagggggaggcatcttcctcaggacatcgggtcctagagggaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccgccacctctagaaagaggtaagggtttaagggatggttggttggtggggtattaatgtttaattacctggagcacctgcctgaaatcactttttttcagg

SEQ ID NO: 56 - DNA, 1567 bp, synthetic construct
  misc_feature 1..1567: note = Description of Artificial Sequence: Synthetic polynucleotide
  source 1..1567: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctaaggaggggggacagggcagggggaggcatcttcctcaggacatcgggtcctagagggaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctataaaaggtgagctcgtttagtgaaccgtcagtccgcctggagacctcgagccgagcggtcgtccgctgcctcctctgtgcgtccgcccagccagcctcgtccacgccgccacctctagaaagaggtaagggtttaagggatggttggttggtggggtattaatgtttaattacctggagcacctgcctgaaatcactttttttcagg

SEQ ID NO: 57 - DNA, 1618 bp, synthetic construct
  misc_feature 1..1618: note = Description of Artificial Sequence: Synthetic polynucleotide
  source 1..1618: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctaaggaggggggacagggcagggggaggcatcttcctcaggacatcgggtcctagagggaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctataaaaggtgagctcgtttagtgaaccgtcagtccgcctggagacctcgagccgagcggtcgtccgctgcctcctctgtgcgtccgcccagccagcctcgtccacgccgccacctctagacacaggtaagtatcaaggttacaagacaggtttaaggaggccaatagaaactgggcttgtcgagacagagaagattcttgcgtttctgataggcacctattggtcttactgacatccactttgcctttctctccacagg

SEQ ID NO: 58 - DNA, 1623 bp, synthetic construct
  misc_feature 1..1623: note = Description of Artificial Sequence: Synthetic polynucleotide
  source 1..1623: mol_type = other DNA
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggacagggcagggggaggcatcttcctcaggacatcgggtcctagagggaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccgccacctctagacacaggtaagtatcaaggttacaagacaggtttaaggaggccaatagaaactgggcttgtcgagacagagaagattcttgcgtttctgataggcacctattggtcttactgacatccactttgcctttctctccacagg

SEQ ID NO: 59 - DNA, 68 bp, synthetic construct
  misc_feature 1..68: note = Description of Artificial Sequence: Synthetic oligonucleotide
  source 1..68: mol_type = other DNA
tataaaaggtgagctcgtttagtgaaccgtcagtccgcctggagacctcgagccgagcggtcgtccgc